(19) United States
(12) Patent Application Publication
(10) Pub. No.: US 2025/0147006 A1
(43) Pub. Date: May 8, 2025

(54) MULTI-DEPTH SPIRAL MILLI FLUIDIC DEVICE FOR WHOLE MOUNT ZEBRAFISH ANTIBODY STAINING

(71) Applicant: THE REGENTS OF THE UNIVERSITY OF CALIFORNIA, Oakland, CA (US)

(72) Inventors: Songtao Ye, Half Moon Bay, CA (US); Chih-Wen Ni, Castro Valley, CA (US)

(73) Assignee: THE REGENTS OF THE UNIVERSITY OF CALIFORNIA, Oakland, CA (US)

(21) Appl. No.: 18/939,842

(22) Filed: Nov. 7, 2024

Related U.S. Application Data
(60) Provisional application No. 63/596,697, filed on Nov. 7, 2023.

(51) Int. Cl. G01N 33/50; C12M 1/00; C12M 1/34; C12M 3/06

(52) U.S. Cl. CPC G01N 33/5014; C12M 23/16; C12M 29/06; C12M 41/46; G01N 33/5082

(57) ABSTRACT

Fluidic systems, devices and methods are provided for separating and sequestering particles from a fluid flow within traps in a fluidic chip is provided. The fluidic platform is particularly suited for parallel live zebrafish embryo studies providing automated zebrafish embryo trapping and flow through culture as well as whole mount zebrafish antibody staining functions. The zebrafish on a chip testing platform uses a chaotic hydrodynamic trapping process to trap and retain zebrafish embryos in a consistent body orientation (i.e., head pointed inward) without any external adjustments. The system and apparatus can also be adapted to be a multifunctional concentration gradient generator (CGG) that can be used to automatically immobilize dechorionated zebrafish embryos and generate chemical gradients for acute fish embryo toxicity (FET) tests.

### CROSS-REFERENCE TO RELATED APPLICATIONS

This application claims priority to, and the benefit of, U.S. provisional patent application Ser. No. 63/596,697 filed on Nov. 7, 2023, incorporated herein by reference in its entirety.

### STATEMENT REGARDING FEDERALLY SPONSORED RESEARCH OR DEVELOPMENT

Not Applicable

### NOTICE OF MATERIAL SUBJECT TO COPYRIGHT PROTECTION

A portion of the material in this patent document may be subject to copyright protection under the copyright laws of the United States and of other countries. The owner of the copyright rights has no objection to the facsimile reproduction by anyone of the patent document or the patent disclosure, as it appears in the United States Patent and Trademark Office publicly available file or records, but otherwise reserves all copyright rights whatsoever. The copyright owner does not hereby waive any of its rights to have this patent document maintained in secrecy, including without limitation its rights pursuant to 37 C.F.R. § 1.14.

### BACKGROUND

### 1. Technical Field

This technology pertains generally to devices and methods for small particle separations and more particularly to devices, systems and methods for automated zebrafish embryo separation and positioning and flowthrough assays.

### 2. Background Discussion

In biomedical research, small animal studies provide distinct advantages over cell lines and tissue studies by allowing researchers to analyze biological processes in the whole organism, a more natural and physiologically relevant environment. Zebrafish (Danio rerio) have become a popular small animal model in the biomedical field due to their high reproduction rate, small size, body transparency, and closer phylogenetic relationship to humans etc. Zebrafish embryos have been used for drug screening, embryotoxicity tests, genetic or tissue functional studies and others.

However, like most bioassays, the zebrafish related procedures are still largely performed manually in well plates or Petri dishes. Experienced personnel need to go through a series of tedious and time-consuming steps including embryo transfer, manipulation, imaging, and retrieving, that greatly limit the throughput and reproducibility of these studies. In addition, the static tests performed on well plates or Petri dishes can be inadequate for some of the zebrafish studies as the fluidic and chemical microenvironments are not fully controlled. Factors such as liquid evaporation, chemical degradation, oxygen depletion, metabolic inactivation, etc. can lead to variations in results and lower the assay consistency in zebrafish studies. Furthermore, high resolution single embryo real-time monitoring is difficult to achieve in the bulk fluidic environment as the movements of zebrafish embryos are not constrained.

Whole mount zebrafish antibody staining (ABS) and in situ hybridization (ISH) using protein and antisense ribonucleic acid (RNA) probes are common molecular staining techniques for detecting the protein and gene localization information on zebrafish. The conventional manual procedure for the whole mount zebrafish molecular staining usually takes hours or days to complete and involves a series of tedious and time-consuming steps. Moreover, the performance and consistency of the procedure is usually skill dependent. To overcome these limitations, automated liquid handling platforms have been developed to perform the assays. These platforms utilize robotic arms or hydraulic systems for automated liquid handling and are usually compatible with well-plates or tubes. Although they can greatly reduce the labor and improve the consistency of the assays, the platforms are not usually affordable in labs with limited budgets.

In 2021, researchers developed a semi-automated liquid handling platform, “Flyspresso”, for the whole mount fruit fly ABS. The Flyspresso platform utilizes a programmed gas-powered hydraulic system for the liquid handling which is portable and is compatible with various staining procedures. This open-source platform provides an economic way for developing customized automated platform for whole organism assays. However, it is not a “sample-in-and-answer-out” platform as the specimens still need to be retrieved and manually mounted for imaging. Moreover, the shaking or repeat pipetting-based mass transfer enhancements in tube and well-plate limit the room for process optimization and time reduction. To address these limitations, one way is to integrate the automated platform with fluidic devices.

Because experimental procedures using zebrafish embryos are largely performed in well plates or Petri dishes, which is time-consuming and requires tedious embryo manipulation and liquid transfer manually, the lack of automation has become one of the major obstacles in zebrafish research and slows down the advancement of related research. Hence, the development of a system enabling a feature of automation of the routine procedures in zebrafish research is urgently needed.

### BRIEF SUMMARY

To fulfill the need for efficient and high through put methods, a milli-fluidic platform is provided that can automate the routine procedures including embryo placing, drug dosage generation, long-term flowthrough embryo culture (i.e., without the need to refresh the buffer), and real-time monitoring (i.e., without the need to retrieve the embryos). Moreover, the platform can also be used for certain zebrafish embryo assays such as immunohistology staining. It has been shown to have an improved performance in both time reduction and result reproducibility compared to the conventional well plate-based methods. Overall, in comparison to the static Petri dish-based methods, the apparatus and systems offer more controllable microenvironments for zebrafish embryo culture as well as a more efficient way for phenotype screening and real-time monitoring.

In one embodiment, zebrafish antibody staining (ABS) is accomplished using a milli fluidic device that can automatically trap and immobilize the fixed chorion-less zebrafish embryos for the whole mount ABS procedure. With just a single loading step, the zebrafish embryos can be trapped by the milli fluidic chip device through a chaotic hydrodynamic trapping process. Moreover, a consistent body orientation (i.e., head pointed inward) for the trapped zebrafish embryos can be achieved without the use of an additional orientation adjustment device.

Furthermore, use of consumer-grade stereolithography (SLA) three-dimensional (3D) printers for device prototyping can be cost effective. Notably, the milli fluidic device has enabled the optimization and successful implementation of whole mount zebrafish Caspase-3 ABS. This device can accelerate the overall procedure by reducing at least 50% of washing time in the standard well-plate-based manual procedure. Also, the consistency is improved, and manual steps are reduced using the milli fluidic device. This works to fill the gap in the milli fluidic application for whole mount zebrafish immunohistochemistry. Collectively, in contrast to the micro- or nano-fluidic devices, the milli fluidic device can be incorporated with an automated liquid handling platform to achieve an automation level of a “sample-in-and-answer-out” procedure.

The fluidic device apparatus and system is based on a fluidic chip that has a central fluid collection reservoir bounded by the inner wall of an arcuate annular channel defined by an outer wall and an inner wall. The inner wall of the channel has a number of particle traps open to the arcuate channel and to the fluid collection reservoir. A pressure differential between the interior of the arcuate outer channel and the interior of the collection reservoir is created such that the particles are drawn into the traps and the typically fill sequentially with the traps closest to the inlets filling first. The arcuate outer channel preferably has a fluid inlet and particle input at one end and is open to the collection reservoir at the other. A negative pressure with the withdrawal of fluid from the collection reservoir creates a fluid flow through the arcuate channel as well as through the traps within the inner channel wall.

The traps are shaped and sized to receive a desired particle within the channel wall. The trap also has a smaller opening at the collection reservoir end to allow fluid to pass through into the collection reservoir. The narrower opening into the collecting reservoir may be of different sizes to control, in part, the suction pressure created by the differential within the trap. The sizes of the opening or nozzles of the traps may also be successively increased or decreased in traps along the length of the channel.

The shape of the trap opening in the channel wall can have edges that are at right angles to the surface of the wall of the interior channel or the opening may have one or more edges that are rounded. One preferred trap shape has a rounded edge and wall on the same side of the device as the fluid/particle inputs. In another embodiment, the particle trap opening has inner walls at right angles to the surface of the channel and a square or rectangular cross-section.

The preferred system and methods for particle separations in a fluid flow has a fluidic trapping chip with one or more fluid and particle inlets and an outlet connected to a pump to allow flow through the chip. The system may also have a loading reservoir and at least one waste container to allow both close-loop and open-loop operations. An optional pulse dampener may also be provided to limit pulsing that may be caused by a peristaltic pump, for example.

Generally, the particles, illustrated with zebrafish embryos, are loaded along with a flow carrier fluid into the arcuate outer channel of the chip through one or more inlets. In one embodiment, the carrier fluid and the selected particles are loaded separately into the fluidic chip. The carrier fluid in the fluid collection reservoir at the center of the chip is withdrawn through an output by the pump thereby creating a fluid flow through the outer channel, traps and fluid collection reservoir of the chip. The structure of the outer channel and traps create a pressure differential between the channel and the central collection reservoir. Particles flowing through the channel are drawn into the traps and maintained by the flow of fluid through the traps to the collection reservoir.

The resistance to fluid flow through an individual trap increases with the trapping of a particle so that other particles within the flow of the channel will flow further down the channel to successive traps. The flow of particle containing fluid through the chip and out of the outlet may also be recirculated to so that each trap is filled with a particle. The particle containing output flow may also be directed to a second chip or more in more complex system embodiments.

Once filled with particles, the fluid flow through the chip may be adjusted to maintain the particle within the trap while reducing stresses that may be experienced by the particle from strong flows or pressures within the trap. In addition, the temperature and composition of the carrier fluid may be controlled and optimized for particle survival, if the trapped particle is alive.

Further aspects of the technology described herein will be brought out in the following portions of the specification, wherein the detailed description is for the purpose of fully disclosing preferred embodiments of the technology without placing limitations thereon.

### BRIEF DESCRIPTION OF THE DRAWINGS

The technology described herein will be more fully understood by reference to the following drawings which are for illustrative purposes only:

FIG. 1 is a schematic depiction of a milli fluidic pumping system suitable for zebrafish embryo trapping and staining or other particle separations according to one embodiment of the technology. The arrows indicate the flow direction during close loop perfusion.

FIG. 2 is a schematic depiction of a design layout for a zebrafish-on-a-chip (ZOC) design with trap configurations that are suitable for trapping unhatched zebrafish embryos according to one embodiment of the technology. Trap dimensions indicated on the figure are a non-limiting working example of this embodiment of the chip design.

FIG. 3 is a schematic depiction of a design layouts for a zebrafish-on-a-chip (ZOC) design with trap configurations that are suitable for trapping dechorionated zebrafish embryos according to another embodiment of the technology. Trap dimensions indicated on the figure are a non-limiting working example of this embodiment of the chip design.

FIG. 4 is a schematic illustration of zebrafish embryo trapping due to due to hydrodynamic suction forces drawing the zebrafish embryos into the traps. The hydrodynamic suction forces are created in the channel and traps by a pressure differential created between the exterior channel and the interior suction chamber.

FIG. 5 is an illustration of a heatmap for the ZOC when perfusing at 20 ml/min with a flow restrictor add-on according to an embodiment of the technology.

FIG. 6 is a schematic circuit illustration of the hydrodynamic particle trapping where particle is drag towards the trapping channel when Q_{M}<Q_{T}.

FIG. 7 is a schematic circuit illustration of the hydrodynamic particle trapping where following particles bypass the trapping channel when Q_{M}>Q_{T}.

FIG. 8 is a schematic circuit illustration showing volumetric flowrate and hydraulic resistance relationships in a parallel trapping configuration according to one embodiment of the technology.

FIG. 9 is a comparison of manual steps and procedure time between the conventional well plate-based method (Top) and ZOC-based method (Bottom) according to an embodiment of the technology.

FIG. 10A is a schematic illustration of one system with arrows indicating flow directions when using the peristaltic pump or a syringe pump during concentration gradient generation.

FIG. 10B is a table showing the operation modes and corresponding apparatus states of the apparatus shown in FIG. 10A.

FIG. 11 is a schematic depiction of a fluidic chip for use in the system of FIG. 10A that is suitable for zebrafish embryo trapping and staining or other particle separations according to an alternative embodiment of the technology. This embodiment has an inner wall inlet and an outer wall inlet and capable of dynamic concentration gradient generation (CGG) for acute FET tests.

FIG. 12 is a graph depicting a zebrafish embryo trapping performance comparison between the design shown in FIG. 3 and the CGG design shown in FIG. 11. The dashed circle indicates the embryo loading inlets of each. As shown, the overall trap occupation rate bar graph for design II (N=16, error bar±SD) and CGG (N=24, error bar: ±SD) and head inward rate line plot for design II (N=16, error bar: ±SD) and CGG (N=24, error bar: ±SD). The configuration difference between designs is indicated at the bottom of the figure. The trap occupation frequency distribution of the design of FIG. 3 was (N=16) and the trap occupation frequency distribution in the design of FIG. 10A was (N=24).

FIG. 13 is a plot of simulated steady state dye or caffeine concentration distribution in the traps of the CGG chip shown in FIG. 11.

FIG. 14 is a plot showing a steady state caffeine concentration distribution in the traps of CGG (N≥3, error bar: ±SD).

FIG. 15 is a schematic illustration of an example schedule for a zebrafish embryo acute caffeine overdose study method.

FIG. 16 is a plot of zebrafish embryo endpoint screenings in a conventional 24-well plate. The zebrafish embryo survival rate after 2-hour caffeine treatment and 24-hour recovery in a well plate.

FIG. 17 is a plot of the tail curvature occurrence rate at different caffeine concentrations in day 1 and day 2 screenings (N≥4, error bar: ±SD).

FIG. 18 is a plot of zebrafish embryo endpoint screening in the CGG design showing tail curvature frequency distribution in the traps of CGG at different steady state concentration gradients (N=6, error bar: ±SD).

FIG. 19 is a schematic illustration of a system of CGGs connections using 2-way flow splitters to create concentration gradients according to one alternative embodiment of the technology.

### DETAILED DESCRIPTION

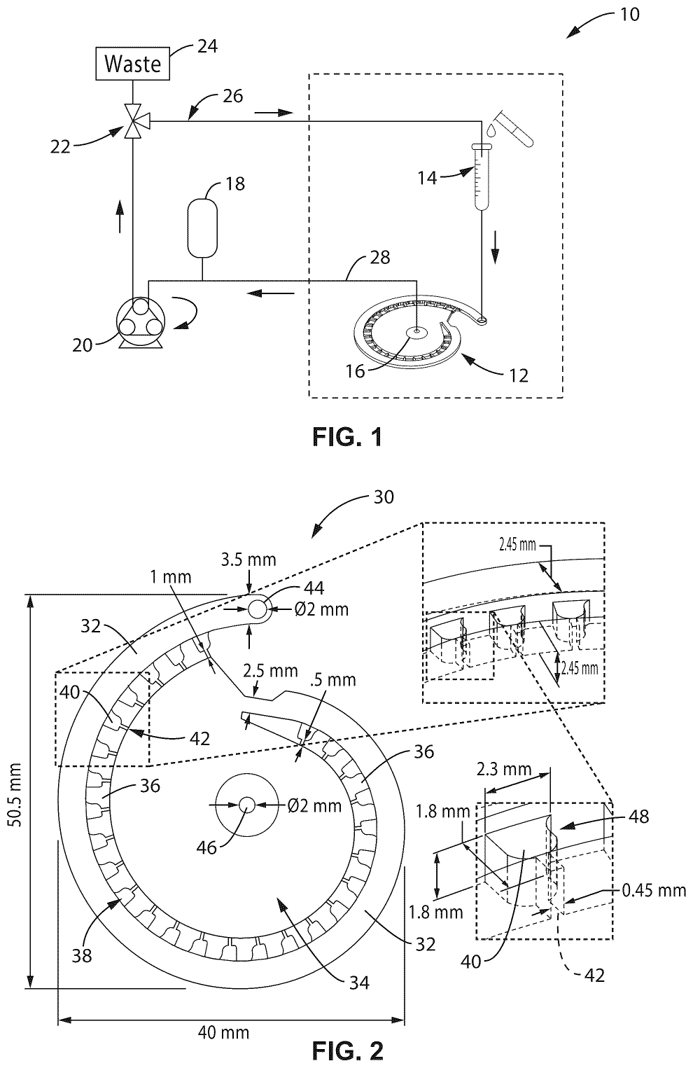

Referring more specifically to the drawings, for illustrative purposes, devices, systems and methods for particle separation and sequestration are generally shown. Several embodiments of the technology are described generally in FIG. 1 to FIG. 19 to illustrate the characteristics and functionality of the zebrafish embryo trapping apparatus and methods. It will be appreciated that the methods may vary as to the specific steps and sequence and the systems and apparatus may vary as to structural details without departing from the basic concepts as disclosed herein. The method steps are merely exemplary of the order that these steps may occur. The steps may occur in any order that is desired, such that it still performs the goals of the claimed technology.

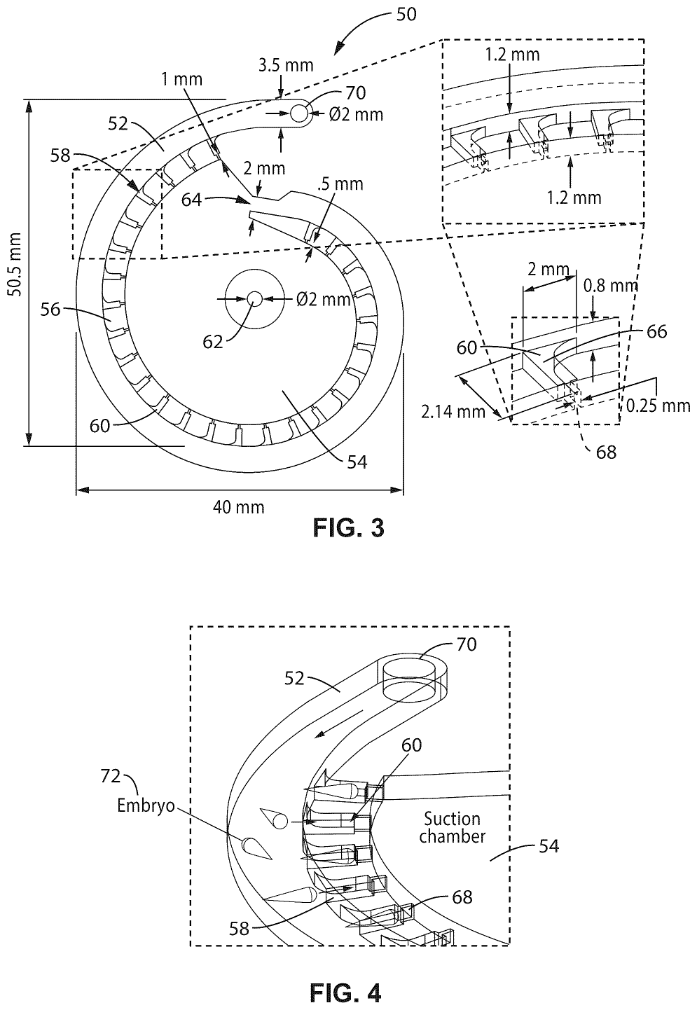

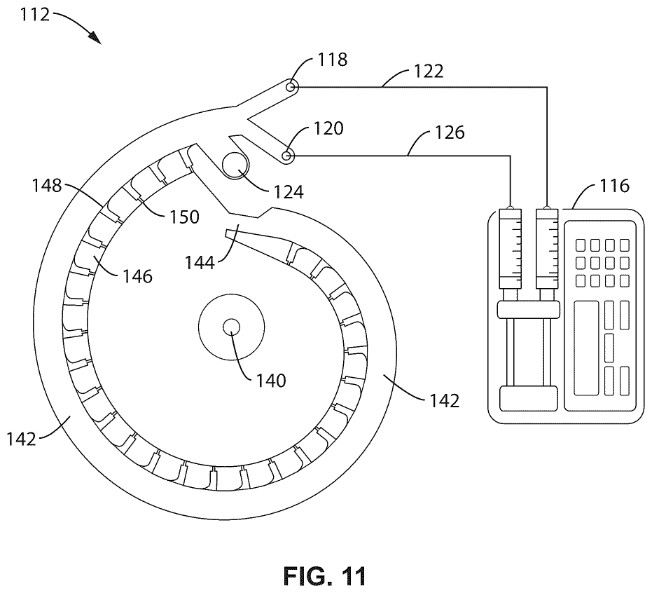

Two example zebrafish-on-a-chip (ZOC) trap configurations for automated zebrafish embryo trapping are described and illustrated in FIG. 2 and FIG. 3. An alternative chip configuration adapted to be a concentration gradient generator is shown schematically in FIG. 11. More particularly, the two ZOCs devices automatically trap the dechorionated and unhatched zebrafish embryos permitting flowthrough culturing. The design and development processes of these two ZOC devices are detailed below. Moreover, the feasibility of performing live zebrafish embryo assays using these two ZOCs are demonstrated and evaluated. Although illustrated with the use of zebrafish, those of ordinary skill in the art will know and understand that any type of fish or fish embryo may be used as human disease models, such as medaka or Japanese rice fish (Oryzias latipes). Likewise, it will be understood that the fluidic chip trap may be configured and used for trapping many different types of particles from a carrier fluid flow that may or may not be alive.

Turning now to FIG. 1, a conceptual system 10 using a fluidic chip 12 for trapping and retaining particles is shown schematically. An optional insulated temperature controlling container is represented by a dashed line. In this general embodiment of the system 10, the fluidic chip 12 has an input 14 of carrier fluid and particles, and an output 16 that is fluidly connected to a pump 20. Optionally, a pulse damper 18 can be disposed in the fluid line 28 between the chip 12 and the pump 20 to limit or restrict any pulses in the fluid flowing through the system from the pump.

In one embodiment, the pump 20 also includes a controller configured to control pump actuation and the flow of fluid through the fluidic system as well as to control the pressure of fluid entering the fluid inlet and the pressure of the fluid exiting the fluid outlet within the collection reservoir of the chip. Thus the controller can create a pressure differential between the arcuate channel and the central fluid collection reservoir of the fluidic chip 12.

In another embodiment, the pump 20 also includes a fluid heating element, a temperature sensor and a fluid temperature control circuit to allow control over the temperature of the fluid flowing through the fluidic circuit and the fluidic chip 12.

The fluidic circuits of the system can be either closed or open. In the embodiment shown in FIG. 1, the system 10 is a closed circuit and the fluid from the pump can be directed through valve 22 to either waste 24 or back through an input fluid line 26 to the input of the chip 12 to provide either clean fluid or to recirculate fluid with particles to the chip to ensure full trap filling.

The fluid chip structure with two variations in trap designs is shown in greater detail in FIG. 2 and FIG. 3. The dimensions and trap number that are shown in the figures are illustrative and not essential features. In the first embodiment of the chip 30 shown in FIG. 2, the outer arcuate channel 32 is defined by an outer wall and an inner wall 36. The inner wall 36 separates the channel 32 from the central interior reservoir suction chamber 34 and the channel is generally annular or spiral shaped. The inner channel wall 36 has a number of particle traps 38 that are shown in greater detail in the exploded detailed views of FIG. 2 and FIG. 3.

The individual traps have a trap chamber 40 open at one end to the channel 32 that is sized to receive the desired particle. The trap chamber 40 is also open to the central fluid collection reservoir 34 (suction chamber) with a smaller opening 42 that may be a slit, duct or channel in shape. The channel or nozzle opening 42 may range in size and is sized to allow a flow of fluid from the arcuate channel 32 through the trap chamber 40 and nozzle opening 42 into the central suction chamber 34. Preferably, some flow through the trap 38 is maintained even when a particle has been trapped in the trap chamber 40.

The opening and walls of each trap chamber 40 may be at right angles to the surface of the inner wall 36 of the arcuate channel 32. In the embodiment shown in FIG. 2, one edge and wall of the opening is at a right angle while the opposing wall 48 and edge is curved or “S” shaped. This rounded edge to the opening of the trap 38 is on the edge opposite of the direction of flow of the fluid through the arcuate channel 32 in this embodiment. The rounded edge in the opening helps to facilitate the initial trapping of a particle from the flow of fluid in channel 32 into the trap 38.

The chip 30 has a fluid and particle input 44 to the arcuate channel 32 to produce a fluid flow into the chip 30. Fluid and/or fluid with untrapped particles is withdrawn from the central suction chamber 34 through the output 46. The withdrawal of fluid from the central reservoir suction chamber 34 creates a negative pressure between the channel 32 and central reservoir chamber 34 and suction through the traps 38.

The dimensions and shape of the traps, channels, channel openings and reservoir can also be tuned to select or optimize the suction forces that are experienced by the particles in the flow or in the traps. An alternative embodiment of a fluidic chip 50 with a variation in the design of the traps is shown in FIG. 3. Similar to the embodiment of FIG. 2, the fluid chip has an outer arcuate channel 52 that has an inner wall 56 that generally defines an inner fluid collection reservoir 54 that creates a suction chamber with removal of fluid from an output. The traps 58 have a body with a trap chamber 60 that is open at one end to the arcuate flow channel 52. The trap chamber 60 is also open to the central fluid collection reservoir 54 by a narrow-slit opening 68. The fluid flow and suction forces within the trap can be adjusted, in part, with the dimensions that are selected for the slit opening 68.

One wall 66 and edge of the trap chamber 60 is rounded increasing the overall size of the opening to the trap chamber 60. The rounded edge 66 of the opening is located on the edge closest to the input 70.

The far end of the arcuate channel 52 is connected to the interior of the central fluid collection reservoir 54 with an opening 64. The dimensions of the opening 64 at the end of the channel 52 can influence the suction and fluid flow rates through the arcuate channel 52 from the suction of fluid from the central fluid collection reservoir 54 through output 62 with a pump.

As shown in FIG. 4, fluid and embryos 72 may be introduced to the arcuate channel 52 through an input 70. Suction generated in the central fluid collection reservoir 54 draws fluid through the arcuate channel 52 and the traps 58 drawing the embryos 72 into the traps 58 and retaining them in the traps with a pressure differential. Suction may also create and help control the flow of fluid along the arcuate channel 52 from its distal open end that is open to the reservoir 54.

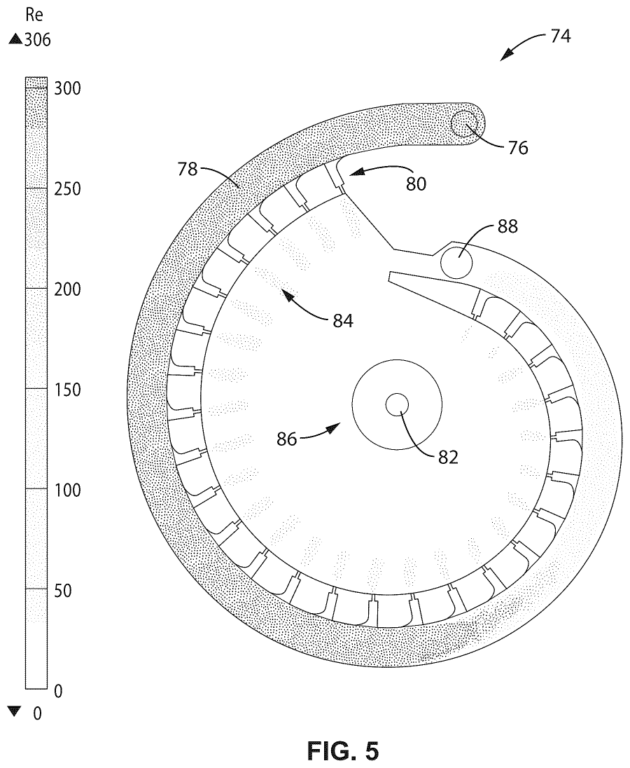

It can be seen that the dimensions of the trap nozzle or opening from the trap chamber to the center reservoir suction chamber allow control or tuning over the suction and fluid flow through the traps. Although the dimensions of the trap openings are uniform in the embodiments shown in FIG. 2 and FIG. 3, the dimensions of the openings can be varied in individual traps along the length of the outer arcuate channel. The selection of trap dimensions of successive traps along the channel can progressively increase or decrease the suction forces of the traps. For example, the length of the trap nozzle opening to the central suction chamber is shown schematically in FIG. 5. In the chip 74, the length of the trap nozzle 80 between the trap and the central chamber was decreased in each trap progressively from the input side of the channel 78 to the chamber side of the channel 78. This feature controlled the fluid flow rate 84 of fluid from the input 76 and channel 18 through the traps and entering the central suction chamber 86 from each trap 80 with a constant fluid removal rate from the output 82 of the central fluid collection reservoir.

In the embodiment shown in FIG. 5, a sphere 88 disposed in the distal portion of the arcuate channel 28 restricts and controls the flow of fluid from the end of the channel that is open to the suction of the interior of the central fluid collection reservoir 86. The reduced or restricted flow from the end of the channel 78 effectively increases the draw forces experienced in each of the traps 80 from the withdrawal of fluid from the reservoir 86 with the output 82.

Accordingly, the chip 74 provides parallel trapping and source-and-sink configurations with particles such as zebrafish embryos dragged into the traps due to hydrodynamic suction force.

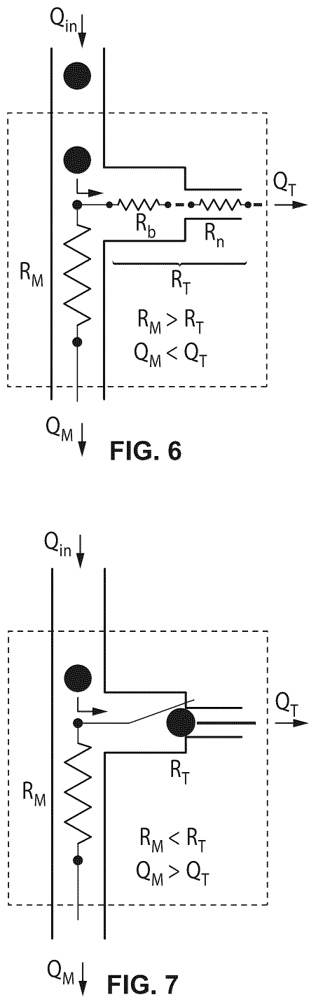

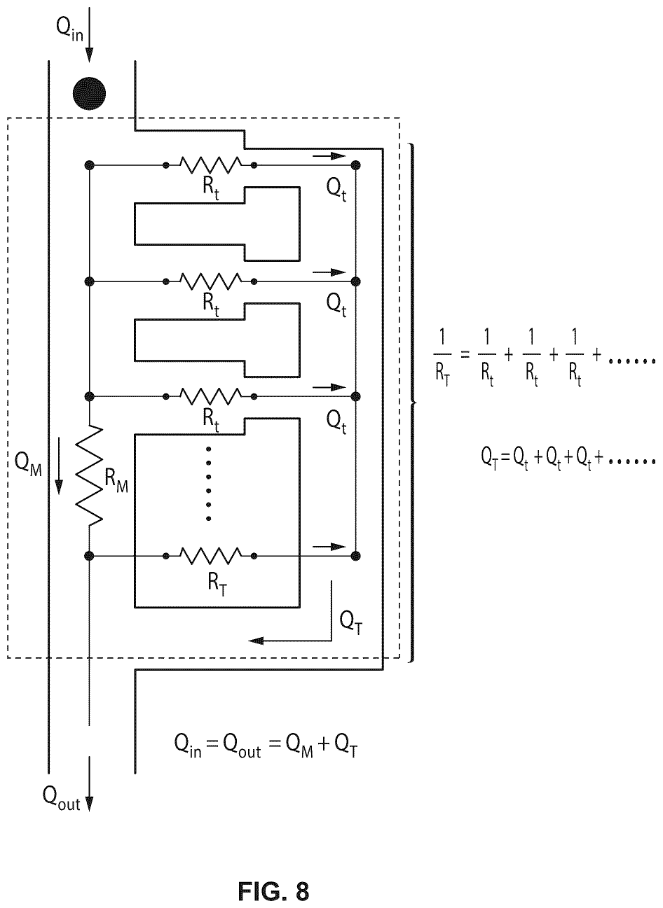

The chip designs utilize hydrodynamic force as the main mechanism for the embryo trapping. To understand the hydrodynamic zebrafish embryo trapping process as well as to determine the appropriate channel dimensions, electric circuit analogy is used to establish resistive flow models for the fluidic chips and shown in FIG. 6, FIG. 7 and FIG. 8. Based on the circuit analogy, the volumetric flowrate, pressure difference, and hydraulic resistance along the channel is equivalent to the current, voltage, and resistance in Ohm's law. Hence, the volumetric flowrate, Q is equal to the quotient of pressure difference, ΔP, and hydraulic resistance, R.

$$Q = \frac{Δ P}{R}$$

Also, the hydraulic resistance for single phase laminar flow along a section of rectangular channel section is determined by both fluidic properties and the channel geometry and is given by

$$R = {\frac{12 μ L}{{h}^{3} w} [ 1 - \frac{192}{{π}^{5}} \times \frac{h}{w} \times tanh tanh ( \frac{π w}{2 h} ) ]}^{- 1}$$

where μ is the dynamic viscosity of the carrying fluid, L, w, and h are the length, width, and height of the rectangular channel section, respectively. A hydrodynamic particle trapping device usually consists of two major channels: (1) an array of trapping channels and (2) a main channel. The steady fluid flow will carry the particles through the main channel and sequentially place single or multiple particles into the traps. The trapping channel's ability to capture the particles from the main channel is based on the relative hydraulic resistances between the trap and the main channel. For instance, particles will be more likely to be drawn into the trap when the hydraulic resistance of the empty trap, R_{T}, is less than the hydraulic resistance of the main channel, R_{M} as shown in FIG. 6.

When a trap is occupied with a particle already, the following particles will bypass the trap as the hydraulic resistance of the occupied trap is now greater than the main channel (FIG. 7). Collectively, the trapping happens when R_{M}>R_{T}.

A hydrodynamic trap design usually involves both body and nozzle (FIG. 6). The trap body or chamber, which usually has dimensions that are close in size to the particle, is used to contain the particle. Whereas the trap nozzle has a width smaller than the particle diameter is used to retain the particle inside the trap. Notably, the dimensions of the trap nozzle are usually used to adjust the hydraulic resistance of the trap in the fluidic device and the rate of fluid flow.

The overall hydraulic resistance of the trap can then be calculated by

$${R}_{T} = {R}_{b} + {R}_{n}$$

where, R_{b} is the hydraulic resistance of the trap body, and Rₙ is the hydraulic resistance of the trap nozzle.

Separately, the volumetric flowrate relationships between trapping channels and main channel also reflect the particle trapping potential and perhaps a more intuitive way to understand the hydrodynamic particle trapping process. In the fluidic channel, the primary force that directs the traveling of the particle is the drag force (FD) which is given by

$${F}_{D} = \frac{1}{2} \times {C}_{D} ρ {Av}^{2}$$

where, C_{D} is the drag coefficient, a dimensionless number that describes the resistance of a moving particle to the carrying fluid. ρ is the carrying fluid density. A is the reference area of the object. ν is the relative velocity between the object and the carrying fluid. As shown in this equation, the level of the trapping force is directly related to the velocity of the carrying fluid. When the channel geometry is considered, the particle drawing ability of the trapping channels can also be estimated by the volumetric flowrate relationship between the main channel and trapping chamber channels. For instance, when the volumetric flowrate in the trap channels (Q_{T}) is higher than the volumetric flowrate in the main channel (Q_{M}), the particle trapping will be more likely to happen and vice versa (FIG. 7).

The preferred two ZOC devices implement a parallel hydrodynamic trapping configuration in which the traps are parallelly arranged and share the same inlet and outlet with the main channel. Because of the simple channel design, this parallel trapping configuration is suitable for milli fluidic chips where the design space is typically limited. The volumetric flowrate, as well as the channel hydraulic resistance relationships, can be described in a simple model.

Based on the continuity equation, the inlet volumetric flowrate (Qᵢₙ) is equal to the sum of the volumetric flowrate in the main channel (Q_{M}) and the overall volumetric flow that goes through the trapping channels (Q_{T}). Similarly, the particle (e.g., zebrafish embryo) is more likely to be trapped when the volumetric flowrate that goes through the trapping channels (Q_{T}) is greater than the volumetric flowrate that goes through the main channel (Q_{M}). Or in another words, the overall volumetric flowrate that goes through the traps must be greater than 50% of the inlet flowrate to let the trapping happen:

$${Q}_{T} > {Q}_{M} = {Q}_{in} - {Q}_{T} ⇒ {Q}_{T} > 0.5 \times {Q}_{in}$$

The above equations explain the criteria for hydrodynamic particle trapping under steady state conditions. However, the particle trapping is a dynamic process in which the hydraulic resistances as well as the volumetric flowrates in the channels are continuously changing as the trapping channels keep being occupied by the particles. Moreover, this dynamic trapping process can be complicated and even chaotic for zebrafish embryos as their substantial volume constantly disrupts the fluidic field when traveling in the milli scaled channels. For this, the fluidic field cannot be assumed to be unchanged (i.e., quasi-steady state) throughout the process. Also, the zebrafish embryos are likely to experience more embryo-embryo and embryo-wall interactions in the channel that is not much bigger than their sizes which makes the embryo trapping prediction even harder. Hence, the dynamic trapping process of zebrafish embryo cannot be easily simulated by a single Lagrangian particle tracing model.

To simplify the description of the dynamic trapping and estimate the overall embryo trapping potential of the ZOCs, the initial (i.e., all traps are empty) and final states (i.e., all traps are occupied) of the trapping process are selected and analyzed as they are the only two steady state moments that are certain in this complicated dynamic process. For parallel trapping configuration, it can be easily understood that the overall volumetric flowrate that goes through the traps drops as more traps are occupied during the trapping (i.e., the overall hydraulic resistance of traps increases when more embryos are captured). To maintain a sufficient embryo drawing potential in the ZOCs even at the late stage of the trapping process, the overall volumetric flowrate that goes through the traps (Q_{T_final}) needs to be kept around or greater than 50% of the inlet flowrate (Qᵢₙ) at the final state.

### At the Final State:

$${Q}_{T \_ final} {\sim}^{>} 0.5 \times {Q}_{in}$$

The above equation can estimate the overall embryo trapping ability of the ZOC, however, to evaluate the embryo trapping smoothness as well as to predict the usage (i.e., embryo occupation potential) of specific traps, individual trap's embryo drawing ability need to be investigated. Because the movements of the zebrafish embryo are directed by the hydrodynamic drag force described above and the embryos usually have a very closed set of physical properties (i.e., density, and shape), the embryo trapping index (ETP) is used to evaluate the embryo drawing ability of individual traps

$$ETP = \frac{{V}_{t}}{{V}_{m}} \times 100 \%$$

where, Vₜ is the average linear velocity towards the trapping channel, and Vₘ is the average linear velocity at the near main channel section.

For a smoothed trapping, the free embryos need to have the momentum to travel in the ZOC throughout the process without being stopped due to flowrate decreasing in the main channel. Therefore, the inlet flowrate selection as well as the flowrate attenuation rate need to be considered when operating and designing the ZOC with parallel trapping configuration. Moreover, the embryos should not all rush into specific traps during the trapping which could lead to channel blockage in the ZOC.

In one embodiment, the embryo drawing potential of all empty traps needs to be kept at a closed level throughout the trapping process or in another words, no empty traps should have outstanding embryo drawing ability compared to the rest of empty traps. For this, a closed embryo trapping potential for individual empty traps need to be ensured at the initial state as the trapping potential for all the empty traps drop simultaneously during the trapping process in the parallel trapping configuration (i.e., the relationship of embryo trapping potential among all the empty traps mostly preserves towards the end).

### All Individual Traps at Initial State:

$$ETP ≅ Constant$$

Collectively, due to the substantial volume and mass, zebrafish embryos experience much larger momentum as well as more complex movements than the micro- and nano-particles during the trapping process. Furthermore, it is not feasible to apply the quasi-steady state assumption to the fluid field when trapping zebrafish embryos, as the macroscopic size of the embryo constantly changes the fluid field. Hence, using the Lagrangian method to predict and estimate the zebrafish embryo trapping ability for ZOC is not cost-efficient. Rather, a simple analytic model for embryo trapping potential estimation in the ZOC by targeting the initial, and final states of the zebrafish embryo trapping process can be used.

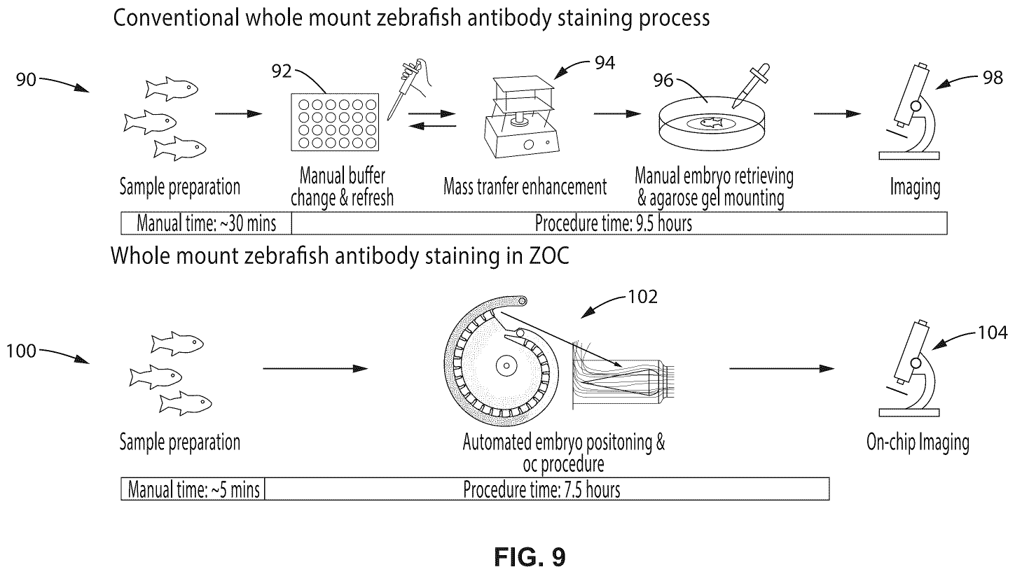

The ZOC-based automatic entrapment of zebrafish embryos is one of the desired features for the ZOC system as it can simplify the embryo transfer/placing processes as well as provide consistent microenvironments for embryo development or treatments. As shown in FIG. 9, the conventional whole mount zebrafish antibody staining process 90 is shown for comparison. The process has the general steps of sample preparation, manual buffer processing 92, mass transfer enhancement 94 and manual embryo retrieving and agarose gel mounting 96 before imaging 98.

In contrast to the conventional well plate and Petri dish-based methods which rely on low-controlled assay conditions and bulk measurements, ZOC systems 100 offer highly controlled experimental conditions for high-throughput and resolution analysis. This allows for assay standardization and automation and the ZOC system provides a device, method, and process 102 before on-chip imaging 104 that allows for “sample-in-and-answer-out.”

Currently whole mount zebrafish in situ hybridization (ISH) is a common molecular staining procedure used in zebrafish research for gene localization information detection. Like the whole mount zebrafish ABS discussed above, the whole mount zebrafish ISH involves a series of washing and staining steps which are labor-intensive, time-consuming, and prone to human error. The development of highly integrated ZOC-based automated systems for whole mount zebrafish ISH significantly reduces the burdens of researchers in performing the assay as well as accelerate the overall zebrafish studies.

Accordingly, ZOC platforms for fully automated zebrafish positioning, testing and analysis can significantly facilitate the zebrafish studies and revolutionize the drug discovery process.

A multifunctional concentration gradient generator (CGG) can be used to automatically immobilize the dechorionated zebrafish embryos and generate chemical gradients for acute FET tests. This allows for the identification of the concentration-dependent sublethal endpoints and conducting high-resolution dose-response analysis which can be used as an alternative tool for the acute FET tests.

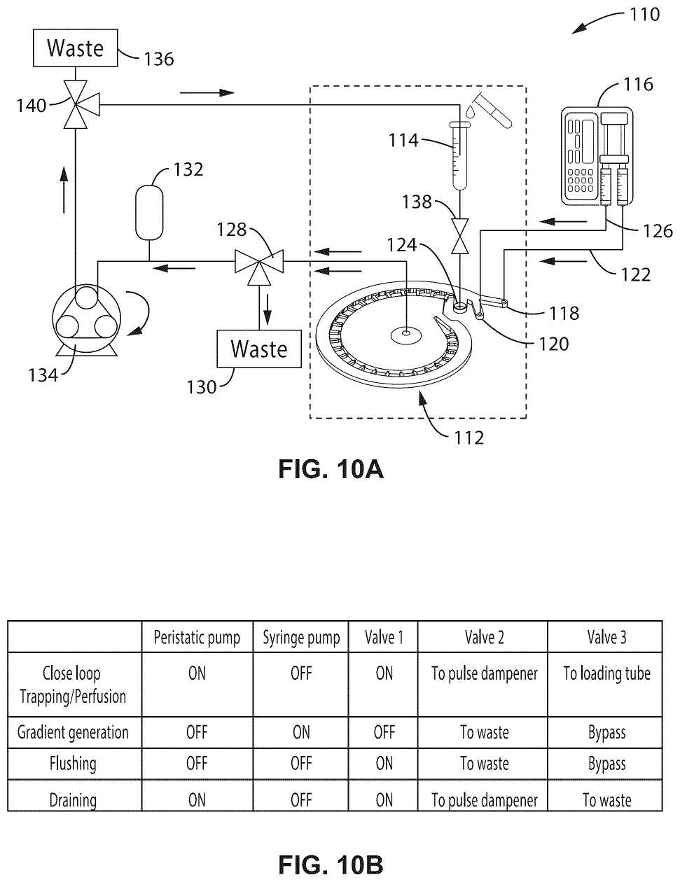

An embodiment of the chip and system 110 to be a concentration gradient generator is shown schematically in FIG. 10A and FIG. 11. As shown in FIG. 10A, the system 110 has a fluid chip 112 with inputs 118, 120 that are coupled with lines 122 and 126 to a syringe pump 116 compatible with syringes for concentration generation purposes. A two-way valve 138 (valve 1) is added to block the loading tube 114 during dosage generation. Also, a three-way valve 128 (valve 2) was placed at the outlet of the CGG chip 112 to switch between the close loop embryo trapping/perfusion and dosage generation modes. This valve 128 (valve 2) is fluidly coupled to a waste 130 and to the pulse dampener 132 that is used to reduce the oscillation and smoothen the flow, and to pump 134.

A second three-way valve 140 (valve 3) is provided between the pump 134 and a second waste 136. This valve 140 is also fluidly coupled to a loading tube 114 input and the two-way valve 138 (valve 1) to close the loop. The three way-valve 140 (valve 3) is used to connect the peristaltic pump 134 outlet, a loading reservoir 114, and a waste container 136 to allow both close-loop and open-loop operations with the system. Pump and valve functions are also shown in the table of FIG. 10B.

For the dynamic chemical concentration gradient generation, a Y branch is added at the main channel and shown in greater detail in FIG. 11. The fluidic chip 112 has an arcuate outer channel 142 that exits into with opening 144 into an internal suction chamber with outlet 140. The inner wall 146 of the channel 142 defines the suction chamber and 148 has traps 148 open to the channel 142 on one end and to the suction chamber through a nozzle opening 150 at the other.

To further improve the trap usage and integrate with the new concentration gradient generation feature, the inlet 124 for the zebrafish embryo buffer loading may also be moved to the trap side of the main channel 142 (i.e., inner wall 146 side) as shown in FIG. 11.

In this configuration, the syringe pumps 116 are connected to one inlet arm with line 122 through inlet 118 and the other inlet arm with line 126 through inlet 124 to deliver different materials. After the zebrafish embryos are immobilized inside the traps, two miscible buffers with different chemical concentrations can be perfused through the two inlets 118, 120 (i.e., inner wall inlet and outer wall inlet) at the Y branch. The two miscible streamlines then mix along the main channel 142 to create various concentration levels inside the traps 148 of the chip 112. At steady state, the concentration levels are stable inside the traps 148 creating steady microenvironments for the acute FET.

The technology described herein may be better understood with reference to the accompanying examples, which are intended for purposes of illustration only and should not be construed as in any sense limiting the scope of the technology described herein as defined in the claims appended hereto.

### Example 1

To demonstrate the breadth and functionality of the fluidic chip designs and methods, the system shown in FIG. 1 and fluidic chips of FIG. 2 and FIG. 3, designated design I and design II, were synthesized for testing. A consumer-grade LCD SLA 3D printer was used to assist the device prototyping. Briefly, the negative mold of the ZOC is first printed by the LCD SLA 3D printer, and then used for PDMS soft lithography to make the PDMS-glass device.

The two ZOCs designs produced had a spiral parallel trapping configuration to automatically trap and immobilize the unhatched and dechorionated zebrafish embryos. The spiral ZOC design contained 3 major functional parts: a spiral main channel, an inner suction chamber, and 26 trapping channels that interconnect the main channel and inner chamber. When operating, the inner chamber, when connected to a peristaltic pump, provides a negative pressure to draw the fluids from the main channel via the traps.

In one fabrication, the trap channels and main channel had different heights to shorten the mass transfer distance in the traps and to prevent bubbles from entering the traps. Also, the main channel for both designs had a width of 3.5 mm which allows multiple embryos to travel simultaneously and let individual embryos freely self-rotate.

Because the unhatched zebrafish embryo has a larger overall size than the dechorionated zebrafish embryo, the overall channel height as well as the internal volume of the traps for design I is greater than design II. The internal volume for the trap body in design I and design II are about 7.4 μl and 2 μl, respectively which allows only 1 embryo per trap. After trapping, the zebrafish embryo partially blocks the trap nozzle ensuring a flowthrough environment. Here, the trap nozzle length is used to regulate the flow distribution in the trap and main channels. In both ZOCs, the length of trap nozzle decreases from 1 mm for the first trap (i.e., trap closest to inlet) to 0.5 mm for the last trap (i.e., trap at the end of main channel) at a rate of 0.02 mm/trap along the main channel.

For zebrafish embryo trapping, this configuration ensures a closed hydrodynamic trapping potential at each trap for the purposes of maximizing the usage of the traps and smoothing the trapping process. In addition, this nozzle length changing configuration is also important to minimize the “procedure lagging” (e.g., mass transfer rate differences) due to the fluid velocity drop in the parallelly arranged traps (i.e., slow down the velocity dropping).

For both ZOCs, the traps are arranged parallelly along the spiral main channel and the inner chamber provides a relatively constant downstream pressure. In a simplified circular diagram, the inner chamber can be treated as the ground in the ZOC. This source-and-sink configuration eases the later fine-tuning as the distribution of hydrodynamic trapping force (i.e., proportional to the pressure drop) is only dependent on the hydraulic resistance of the traps and can be adjusted by modifying the trap nozzle size (i.e., R_{T}=R_{b}+Rₙ).

However, to examine if the zebrafish embryo can be smoothly delivered into the traps as well as to evaluate the potential trap usage rate in the ZOCs, the individual trap's embryo drawing ability was analyzed. The main embryo trapping force in the two ZOCs is the hydrodynamic drag force which is directly proportional to the square of relative velocity between the fluidic flow and moving embryo. During the embryo trapping in the ZOCs, the zebrafish embryo experiences the hydrodynamic drag forces from the crossflow viz. along the main channel and towards the trap channel. Here, the fluidic velocity ratio between the trap channel (Vₜ) and its near main channel section (Vₘ) is used as an index to evaluate the individual trap's embryo drawing ability (i.e., ETP=Vₜ/Vₘ×100%).

For a smoothed embryo trapping process, the zebrafish embryos should not rush into specific traps and the main channel velocity dropping should not be too drastic as these may lower the usage of other traps and lead to main channel blockage. For these purposes, in one example, the nozzle length design in the two ZOCs was changed (the nozzle length for individual traps decreased from 1 mm to 0.5 mm along the spiral main channel). To see how the changing nozzle length affects the flow in the main channel and trapping channels, as well as the embryo trapping index (ETP), chips with different nozzle lengths were compared with chips with all nozzles having the same length of 0.5 mm.

According to the initial state CFD simulations (i.e., all traps are empty), when the changing nozzle length design is not employed in design II, the middle range traps would have outstanding ETP indicating the embryos are more likely to rush into the middle traps during trapping. Moreover, compared to the changing nozzle design, the flow distribution among the parallelly arranged traps is steeper (i.e., drop more quickly), and the flow attenuates more rapidly at the main channel when all the nozzles have the same length.

These results indicate the changing nozzle design allows the ZOC to have a more evenly distributed embryo trapping ability among individual traps and the embryos can maintain a certain level of momentum to loop inside the main channel. In addition to the embryo trapping smoothness improvements, this configuration can also minimize the “procedure lagging” (i.e., delay in mass transfer) in the ZOCs as the trapping channel flowrate distribution is flatten after adjusting the trap nozzle length.

Separately, since the parallel hydrodynamic trapping configuration is used in the ZOC designs, the fluidic velocity levels are expected to change simultaneously in the channels during the embryo trapping. The CFD simulations showed that the embryo trapping potential relationship among all the empty traps would mostly be preserved to the end of the trapping process.

The initial steady state velocity maps for the two ZOC designs could also be verified. The initial state velocity maps indicate that the ETP (i.e., velocity ratios between the trapping channels and local main channel sections) in design I and design II were kept at about 46.61±4.6% and 43.12±4.07%, respectively.

According to the CFD simulation results, the changing nozzle length configuration used in the two ZOC designs allow all the traps to have a closed embryo drawing ability, and therefore can ensure a smoothed trapping process as well as a high trap usage rate. Final configurations for the two ZOCs were based on both the CFD simulations and validation experiments.

### Example 2

To validate the fluidic chip designs for particle trapping, the two designs shown in FIG. 2 and FIG. 3 produced in Example 1 were evaluated. Embryo culturing and imaging of the trapped zebrafish was also validated and compared to conventional Petri-dish imaging.

In one example, 4 hpf unhatched zebrafish embryo and 24 hpf dechorionated zebrafish embryo were used in the embryo trapping validation tests for design I and design II, respectively. Because design I has a larger channel dimension than design II, the trapping flowrate used in design I was higher than design II. Here, the embryo trapping flowrate used in design I and design II are set to be 20 ml/min and 10 ml/min, respectively. To maximize the usage of the traps, the gravitational force was also introduced to assist the trapping. At the late stage of the trapping (when most traps were occupied), the device was titled towards the remaining empty traps. Based on the validation test results the average trap occupation rate can reach 86.54±6.57% (N=6) and 93.03±4.3% (N=16) for design I and design II, respectively.

Furthermore, the trap usage distribution showed that the first couple of traps were usually skipped by the embryos even with the help of the gravitational force. This was likely due to the high main channel velocity near the inlet which gives the embryo less deviation time to migrate towards the inner wall traps.

In design II, the validation experiments showed that about 92.88±4.44% of trapped embryos had their heads pointing inward after the trapping. This orientation consistency can be contributed to the coupling effects of the body shear stress and the hydrodynamic suction on the dechorionated zebrafish embryo. Briefly, when traveling in the main channel, the head of the cone-shaped zebrafish embryo experiences higher shear stress than its narrowed body. At the same time, the hydrodynamic suction draws the zebrafish embryo towards the inner wall where the traps are located. As such, the head of the zebrafish embryo was more likely to rotate towards the inner wall and then be dragged into the trap.

Thereafter, the portable device can be transferred to microscope stations for imaging. This feature is not possible with current methods or similar ZOCs. The potential usages for this feature in zebrafish studies such as metabolism analysis, hypoxia studies, and toxicity tests may be possible.

To stably transfer the device to various imaging platforms without disturbing the embryos' positions, the buffer was drained out of the two ZOCs after the trapping. Due to the presence of Laplace pressure, the holdup volume of the remaining buffer forms droplets inside the traps. These droplets can wrap around individual zebrafish embryos forming isolated chambers. The encapsulated zebrafish embryos are unlikely to be disturbed by actions such as device unplugging and relocation.

After the trapping, the zebrafish embryos only partially block the traps in the ZOC and allow the medium or drug to continuous perfusion through the traps. This allows the establishment of stable microenvironments for the zebrafish embryo which is important for assays such as drug screening and embryonic toxicity studies. ZOC flowthrough systems are feasible for long-term zebrafish embryo culture. The perfusion flowrate should be maintained at certain levels to ensure a high embryo survival rate. Zebrafish embryo culture time can be based on the geometry and size of the traps as the embryo may escape due to the hatching and the immobilized embryo will eventually outgrow the size of the trap. In one example, the 4 hpf unhatched and 24 hpf dechorionated zebrafish embryos were loaded and immobilized in design I and design II, respectively.

After embryo immobilization, the ZOCs were close loop perfused with E3 buffer at 10 ml/min. For the control experiment, a 60 mm Petri dish filled with 20 ml E3 buffer was used for the static zebrafish embryo culture (26 embryos per Petri dish). The temperatures for both ZOC-based and static Petri dish-based zebrafish embryo culture were kept around 28.5° C. and the endpoints were measured every 24 hours.

The zebrafish embryo culture in design I carried on for 120 hours. The results showed that the zebrafish embryo survival rate in design I was comparable to the static Petri dish culture. The initial survival rate drops between 4 hpf and 24 hpf were contributed by the unfertilized eggs. Slight decreases in embryo survival rate in the Petri dish culture were observed after 24 hpf, while the embryo vitality in design I maintained at a constant level throughout the experiment. Despite no noticeable embryonic development delay was observed in the zebrafish embryos cultured in design I, the hatching was found significantly postponed as less than 10% zebrafish embryos have successfully hatched after 120-hour culture in design I.

Zebrafish embryos immediately began hatching after disengaging the perfusion at 72 hpf. Also, for the hatched zebrafish embryo, the traps did not constrain their movements or prevent them from escaping during the perfusion. The embryo survival rate in design I is comparable to the Petri dish static control with no morphology abnormality cultivated in the flowthrough environment. Yet, the shear stress exerted by the fluid flow can be an environmental stress that affects the zebrafish hatching. Based on the CFD simulation, the shear stress applied on the zebrafish embryo drops in the parallel arranged traps along the main channel. When perfused at 10 ml/min, a maximum shear stress of 0.0659 Pa was found at the top surface of the embryo located in the first trap. This maximum shear stress is close to the 0.0613 Pa that some researchers have found can cause the hatching delay. In one embodiment, applying lower flowrates during embryo culture with design I for zebrafish embryo studies before 72 hpf in which most embryos are still in chorion resulted in more hatchlings.

Dechorionated 24 hpf zebrafish embryos were used with design II. Like the experiments conducted for design I, the zebrafish embryo survival rate in design II was also found close to the Petri dish static embryo culture. However, with the current trap dimension, the zebrafish embryos were found to already outgrow the trap at around 48 hpf, despite still being immobilized by the hydrodynamic suction force. Furthermore, the size increase of embryo may raise bias for some long-term live zebrafish assays as the trap hydraulic resistance would increase and the fluid field in the design II would vary over time. Because of these, the ZOC-based zebrafish embryo culture in design II was only carried on for about 48 hours to minimize the impacts from the size increase of zebrafish embryos.

For imaging quality, embryo images taken in the ZOC device have similar clearness and resolution compared to embryo images taken in the Petri dish when using stereomicroscope with a CMOS USB camera under normal light. The CCD camera was also used to take fluorescent images for the 48 hpf transgenic zebrafish embryos Tg (kdrl:EGFP). The vascular endothelial cells in the 48 hpf Tg (kdrl:EGFP) zebrafish expressed green fluorescent protein and could be detected under fluorescent microscopy.

In design II, the 48 hpf Tg (kdrl:EGFP) zebrafish embryos were encapsulated in E3 buffer after trapping and then moved to the fluorescent microscope for image taking. For comparison, the 0.5% agarose gel was used to immobilize the zebrafish embryos on the plate. The resolution and contrast of the CCD images for the ZOC immobilized embryos was comparable to the images of agarose immobilized embryos as the intersegmental vessels (ISV) in the zebrafish embryo were visualized in both methods.

Collectively, both ZOC designs showed feasibility for conducting zebrafish embryo studies and have demonstrated convenience in acquiring phenotype-based data. For live zebrafish embryo studies specifically, the time-frame for performing the tests on the ZOC device depended mainly on the geometry and the dimensions of the traps. Also, the impact on embryonic development from shear stress was considered when choosing the flowrate for zebrafish embryo culture. Applying a low shear stress in the flowthrough environment may be best for zebrafish embryo culturing as well as other live zebrafish assays.

In sum, the ZOC design principles for zebrafish embryo trapping (i.e., hydrodynamic trapping) had an over 85% trap usage rate (i.e., trap occupation rate). For design II, the trapped zebrafish embryos showed a consistent body orientation pattern which is valuable for high-throughput imaging and phenotype-based screening. Results of the embryo culture tests indicated that the zebrafish embryos cultivated in ZOCs have a close vitality to the zebrafish embryos cultivated in the static Petri dish. Furthermore, the perfusion flowrate used in the ZOC can be selected such that the shear stress reduces zebrafish embryo hatching delays as well as prevent abnormal postures in the chorion-less zebrafish embryos.

### Example 3

The fluidic chip design and system were further evaluated and validated with whole mount zebrafish Caspase-3 antibody staining (ABS). The whole mount zebrafish Caspase-3 ABS scheme is a well-established assay to detect the level of cell apoptosis in the zebrafish. To induce the Caspase-3 cleavage, UV light, a common environmental stress triggering apoptosis pathway, was applied on the zebrafish embryo. To determine if the whole mount zebrafish ABS can be improved in the flowthrough environment, the whole mount zebrafish Caspase-3 ABS using the ZOC was compared with the results with conventional plate-based manual procedures.

The general whole mount zebrafish ABS procedure normally involves both staining and washing steps. To ensure the antibody can sufficiently bind to the antigen, the staining time is usually kept at an extended level (e.g., overnight). Despite the fact that no systematic study has been conducted to investigate how the fluidic flow can affect the antibody-antigen interaction in the whole mount zebrafish, the studies performed in tubes or well plates suggested that the macromolecular (i.e., antibody and RNA probes) staining usually takes longer in older embryos as tissues becomes denser. The intact tissue of the zebrafish embryo certainly affects the antibody penetration.

Therefore, optimizing the whole organism staining procedure by reducing the staining time may result in a loss of sensitivity. The washing step, on the other hand, is for the removal of the nonspecific binding after the staining step and is crucial for the specificity of the procedure. Targeting the washing steps will be a safer option for the optimization of the whole organism staining as the true signal is ensured with sufficient staining time. Also, the manual buffer refreshing steps can be avoided in the ZOC washing as the wash buffer is circulated in a close loop. For these, the washing steps were targeted in the whole mount zebrafish Caspase-3 ABS procedure for optimization.

Two flowrates viz., 10 ml/min and 20 ml/min were used to perform 30 mins, 60 mins, 90 mins, and 120 mins washings after each staining step. To ensure the consistency and sufficient Casapse-3 binding, the flowrate and time for the two staining steps were kept constant at 10 ml/min and 120 mins, respectively. For comparison, the same washing times were tested in the plate-based procedure using a 24-well plate. After the procedure, the Caspase-3 signals were measured from the zebrafish embryos encapsulated by the PDST droplets in the ZOC (i.e., the holdup volume of PDST wash buffer due to the Laplace pressure) and 0.5% agarose in well plate.

The washing process was found to be accelerated by using the ZOC as significant intensity differences were found at 30 mins, 60 mins, and 90 mins between the well plate-based and ZOC-based washings. Also, samples were found to be sufficiently washed at about 60 mins when the washing flowrate was 20 ml/min in the ZOC.

Additionally, a significant intensity difference was found between the two tested washing flowrates at 60 mins which showed that a higher washing flowrate in the ZOC can speed up the washing process. The two tested washing flowrates in the ZOC showed insufficient washing at 30 mins as the intensities for both flowrates were significantly higher than the control (i.e., 2-hour washing in well plate), and no significant intensity differences were found to be distinct between them.

Regarding the image taking environments, the intensity measured from PBDT droplets inside the ZOC has a slightly decreased signal when compared to the intensity measured in the 0.5% agarose gel. This may be due to the power attenuation when the fluorescent laser traveled through the thick PDMS layer (i.e., 6.2 mm) above the embryos. Despite this, the Caspase-3 signal measured from the PDST droplet in ZOC and from 0.5% agarose in well plate showed no significant difference in intensity levels. When the intensity of all the experimental results were normalized and measured by respective means, the consistency of the ZOC-based procedure was found to be higher than that of the manual well plate-based procedure. Also, the consistency of the assay was improved when applying a higher washing flowrate in the ZOC.

Overall, the ZOC-based whole mount zebrafish ABS outperformed the conventional plate-based manual approach by reducing both manual steps and time while increasing the consistency of the results (e.g. FIG. 9). This highlights the benefits of miniaturization and mechanization of zebrafish assays.

The current needs for optimizing and automating the time consuming and labor-intensive procedures of whole mount zebrafish ABS and ISH is still largely unfulfilled. However, the complete procedure of the whole mount zebrafish ABS was performed and optimized on a ZOC system. The whole mount zebrafish Caspase-3 ABS procedure can be accelerated by employing a higher perfusion flowrate, which may also apply to other whole mount ABS procedures.

Embodiments of the ZOC described herein applies the classic hydrodynamic trapping mechanism to trap chorion-less zebrafish embryos in a close loop perfusion system. The described ZOCs show a trap usage rate that is comparable to the previously reported zebrafish embryo trapping platforms, but with a more convenient loading procedure as multiple embryos are allowed to enter the ZOC at the same time. Also, the body orientation preference (i.e., head point inward) found when trapping the cone-shaped fixed zebrafish embryo may be useful in applications that require parallel phenotype comparison. This phenomenon may also provide some insights for the trapping and sorting of non-spherical particles in other fluidic devices. In addition, the trapped embryos were found to be encapsulated in the droplets after draining out the buffer. The encapsulation of embryos in droplets makes the ZOC portable and allows the ZOC to access various imaging platforms after trapping. This feature, which has not been reported by similar ZOCs, may have potential to be used for applications such as drug screening, metabolite analysis, hypoxia study, and the like.

### Example 4

The fluidic chip and system adaptation as a concentration gradient generator was evaluated with high resolution zebrafish embryo dose-response screening and the system shown in FIGS. 10 and 11. A multifunctional concentration gradient generator (CGG) can be used to automatically immobilize the dechorionated zebrafish embryos and generate chemical gradients for acute FET tests. This allows for the identification of the concentration-dependent sublethal endpoints and conducting high-resolution dose-response analysis which can serve as an alternative tool for the acute FET tests.

The CGG chip design uses a Y-junction to mix the two introduced buffers along the spiral channel and create various concentration levels in the traps. As shown in FIG. 11, the inlet for the zebrafish embryo/buffer loading is moved to the trap side of the main channel (i.e., inner wall) in this fluidic chip design. The height of the main channel and inner chamber was increased to 1.4 mm and the height of the traps is decreased to 0.7 mm for the purpose of preventing bubbles, especially the medium sized bubble, from entering the traps. Also, the nozzle length for the traps has now decreased from 0.8 mm to 0.45 mm at a rate of 0.014 mm/trap along the spiral main channel to compensate for the loss of hydrodynamic suction force due to the height adjustments. After the zebrafish embryos were immobilized inside the traps, two miscible buffers with different chemical concentrations were perfused through the two inlets (i.e., inner wall inlet and outer wall inlet) at the Y branch creating different concentration levels inside of the traps.

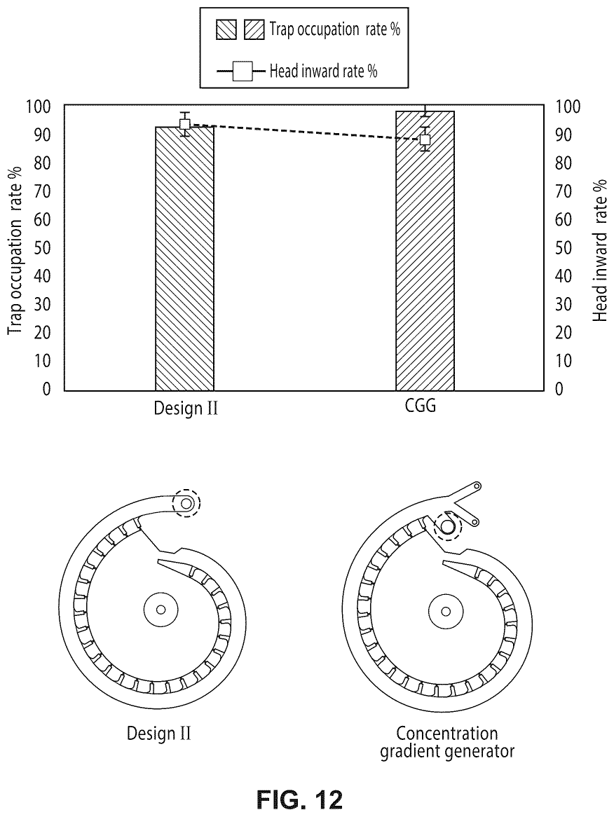

The trap occupation rate of the CGG chip was compared with the design II chip as shown in FIG. 12. The results showed that the trap occupation rate for the CGG can reach 98.4±2.2% (N=24) which is higher than the 93.03±4.35% (N=16) for the design II. Consistent with prior assessments, most of the captured zebrafish embryos had their head pointing inward inside the traps, given an overall head inward rate of 88.08±4.73% (N=24) in the CGG which is slightly lower than the 92.88±4.44% (N=24) measured in design II (FIG. 12). Additionally, after the modifications from design II (i.e., embryo loading inlet location and channel dimensions), the occupation rate of the first trap in the CGG has significantly increased from 6.25% to 79.2%.

Before using CGG for live zebrafish embryo testing, a validation experiment using whole mount zebrafish trypan blue staining was performed to verify the CGG's viability for concentration gradient generation. In this preliminary test, the inner wall and outer wall inlets were perfused with 0.04% Trypan blue/E3 buffer at 25 μl/min and E3 buffer at 125 μl/min, respectively. After the 20-min staining, the parallelly placed fixed zebrafish embryos developed a decreasing trypan blue intensity along the spiral main channel. This indicated that the CGG could treat the embryos with variable chemical concentrations inside the traps, and the 26 concentration levels decreased along the spiral main channel. Furthermore, the gradient can be varied by adjusting the flowrates at the two inlets (data not shown). Trypan blue is a good colorimetric indicator to track the concentration gradient as well as to detect any cell/embryo death events during the live zebrafish embryo test (i.e., trypan blue can only penetrate through dead cells).

One concern about using the CGG for a chemical toxicity test is the treatment time lag. This is because of the dynamic concentration gradient generation process in the CGG. For instance, the zebrafish embryo in the first trap always has the longest treatment time compared to the zebrafish embryos in the downstream traps due to the earliest steady state concentration establishment (i.e., close to the inlets). Therefore, convective-based gradient generation (Pé>1) is preferred for the acute chemical toxicity test as the treatment time differences can be minimized by quickly reaching the steady state concentration levels. Here 7 different flowrate combinations were selected to study the dynamic concentration gradient generation. Specifically, the outer wall inlet is perfused with E3 buffer, and the flowrate (Q_{in_outer wall}) is maintained at 300 μl/min. The inner wall inlet is perfused with 0.04% Trypan blue/testing chemical (e.g., 2 mg/ml Caffeine) mixture and the flowrates (Q_{in_inner wall}) are set to be 10 μl/min, 30 μl/min, 50 μl/min, 60 μl/min, 75 μl/min, 100 μl/min, and 150 μl/min.

To check to see if the dynamic gradient generation is convective mass transfer dominant, the Pé number heatmap was generated using the CFD and mass transfer simulations. The heatmap showed that the Pé number decreases along the spiral main channel before reaching the narrowed section. Since the level of Pé number is dependent on the relative velocity between the two miscible streams, the lowest Pé number should be found at the minimum relative velocity viz. Q_{in_inner wall} equals 150 μl/min (FIG. 40A). When Q_{in_inner wall} is 150 μl/min, the lowest Pé number found at the end of the main channel is around 550 indicating the selected flowrates can ensure a convective-based gradient generation in the CGG. The characteristic length of the main channel was used to calculate the Pé numbers in the CGG. According to the time dependent mass transfer simulations as well as the validation experiments, a stable concentration level inside the traps could be reached in about 10 mins for all the chosen flowrates.

The duration of the zebrafish embryo chemical toxicity tests may be based on both the temporal resolution of the pump and the zebrafish embryo growth rate. A syringe pump compatible with 60 ml syringe may be used. Therefore, the maximum testing window was around 3.33 hours when applying a constant Q_{in_outer wall} of 300 μl/min for the dynamic concentration gradient generation. Also, since the dynamic concentration gradient generation is highly sensitive to changes in fluidic velocity fields, the size increase of the zebrafish embryo would increase the hydraulic resistance inside the traps and thus vary the fluidic velocity field. The 3.33-hour test window is considered suitable for the CGG-based acute FET test as the zebrafish embryo size increase is not that significant and the variations in concentration levels are expected to be minor. According to the Organization for Economic Cooperation and Development's OECD's guideline for the acute FET tests (TG 236), the chemical exposure time for the zebrafish embryo needs to be 96 hours. The acute zebrafish embryo chemical toxicity tests focused on some extreme cases where the sublethal endpoints (e.g., tail curvature, pericardial edema, yolk sac edema, etc.) were developed after short period of chemical exposure (i.e., less than 3.33 hours).

For live zebrafish embryo assays, the shear stress impact to the embryonic development is another factor that was be considered. Although some previously reported ZOCs and flow through systems have shown that the continuous flow is unlikely to cause morphological changes or development abnormalities on zebrafish embryos. Based on the CFD simulations, the maximum shear stress was found on the upper surface of the embryo body inside the trap. When applying the highest overall flowrate (i.e., 300 μl/min+150 μl/min=450 μl/min) for concentration gradient generation, the maximum shear stress can reach approximately 0.021 Pa on the embryo in the first trap. Also, the maximum shear stress is about 0.0835 Pa on the embryos when applied 2 ml/min (i.e., the lowest flowrate for the peristaltic pump) for close loop perfusion. The maximum shear stress level for both operation modes are lower than the maximum shear stress of 0.088 Pa of prior systems which is considered as a low shear stress environment for the zebrafish embryonic development and is expected not to cause development abnormalities for the zebrafish embryos.

The zebrafish embryos are exposed in the steady state concentration gradient for the acute FET test. Based on the mass transfer simulations and validation experiments, the interface of the two miscible streamlines is found to shift towards the inner wall traps along the spiral main channel. This interface shifting is due to the hydrodynamic suction flows that pass through the traps. Besides, the degree of interface shifting is dependent on the flowrate ratios of the two inlets as the “thickness” of the inner wall streamline decreases when Q_{in_inner wall}/Q_{in_out wall} decreases. At steady state, the two streamlines formed “Taichi-like” concentration gradient patterns in the CGG.

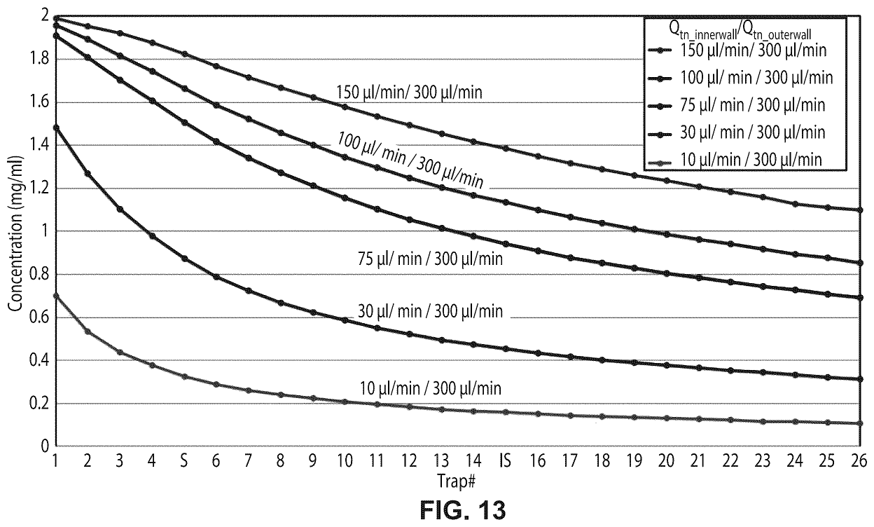

The acute FET test described below selects caffeine as the testing chemical. For the test, the outer wall inlet is perfused with E3 buffer at a constant flowrate of 300 μl/min (Q_{in_outer wall}). To track the caffeine concentration, 0.04% trypan blue/2 mg/ml caffeine mixture is perfused through the inner wall inlet. Five inner wall inlet flowrates (Q_{in_inner wall}) viz. 10 μl/min, 30 μl/min, 75 μl/min, 100 μl/min, and 150 μl/min were selected for steady state caffeine concentration simulations and measurements. Based on the mass transfer simulation, the concentration level and gradient range at steady state depend on the flowrate ratio between the inner wall inlet and outer wall inlet. The concentration level increases while the gradient range decreases when Q_{in_inner wall}/Q_{in_outer wall} increases (FIG. 13). For instance, the concentration in the first trap can reach a minimum of 0.7 mg/ml when Q_{in_inner wall}

$$10 μ l / min ( i . e . , {Q}_{in \_ inner wall} / {Q}_{in \_ outer wall} = \frac{1}{30} ,$$

the lowest ration). Meanwhile, the concentration difference between the first and last trap reaches a maximum of 6.5 times.

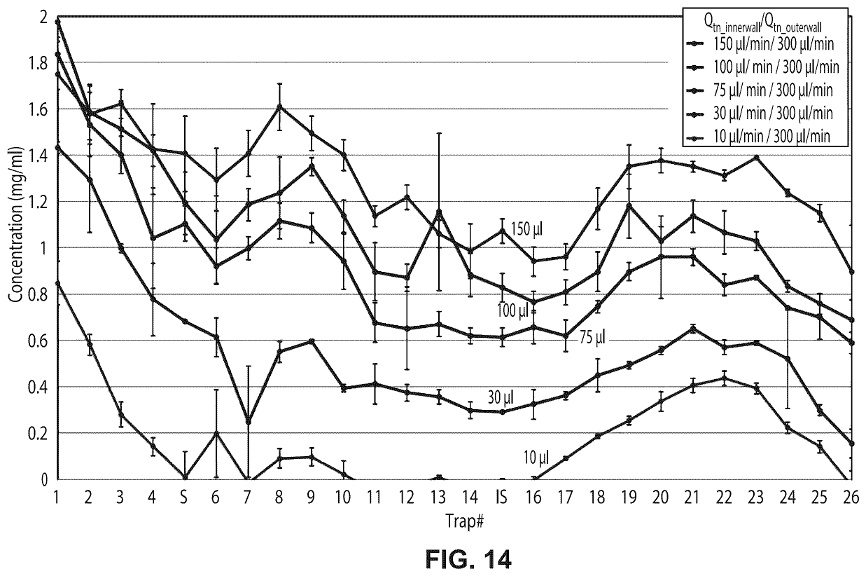

For experimental measurements, the steady state concentration level at each individual trap was estimated by the trypan blue intensity. A MATLAB script was used to obtain the trypan blue/caffeine mixture intensities at each individual trap. The experimental concentration measurements showed similar, yet trembling trends to the simulation results as shown in FIG. 14. The trembling and varied experimental concentration measurements may be due to the limitations of the imaging tools as well as the surrounding environment. The ZOC system was utilized with a USB microscope and an LED light source for imaging. The CMOS digital camera in the USB microscope had poor light sensitivity for the intensity measurement and was susceptible to the noise (i.e., environment light). Moreover, the intensity on the LED light source was found not to be evenly distributed. Thus, to ensure accurate concentration measurements, additional image processing may be necessary to increase the signal to noise ratio. Also, image acquisition using a CCD camera, a stable light source, as well as a low noise environment, may improve the overall concentration measurements.

### Example 5

Acute zebrafish fish embryo toxicity (FET) tests are simple and robust assays in assessing the acute toxicity of chemicals and have been implemented in the zebrafish-on-a-chip (ZOC) platforms for high-throughput endpoint screening. Plate-based acute FET tests for caffeine overdose and toxicity were acquired and compared with the FET tests using the ZOC system platform.

Most caffeine acute toxicity studies follow the OECD guideline TG 236 to test the acute toxicity of caffeine. Despite it being a simple and robust method for assessing acute chemical toxicity, TG 236 has limitations to simulate complex chemical exposure scenarios. In addition, the OCED guideline TG 236 for acute FET test determines the lethality by the 4 core endpoints viz. coagulation of fertilized eggs, lack of somite formation, lack of detachment from the yolk sac, and lack of heartbeat. Because the focus is shifting toward the detection of lethal endpoints, the concentration-dependent sublethal endpoints are sometimes neglected in the acute FET tests which can be critical in understanding the toxic effects for certain chemicals.

Here, the objective was to investigate the instant effects of caffeine overdose as well as how these effects change during the recovery period. To restore a more realistic caffeine consumption scenario, a much shorter zebrafish embryo caffeine treatment time was used compared to the standard acute FET test procedure. Furthermore, most of the zebrafish embryos were exposed to an extensive amount of caffeine in this short period to simulate the caffeine overdose. Most importantly, to capture the instant effect of caffeine overdose, the temporal feature of the sublethal endpoint was considered in the screening and the sublethal endpoints are ranked by their timing of occurrence during the test.

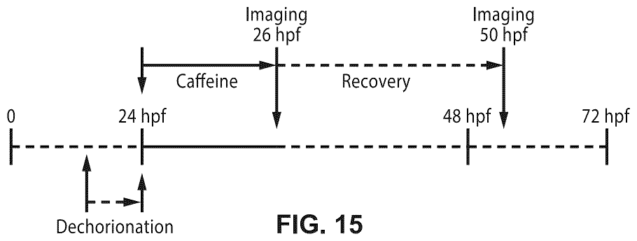

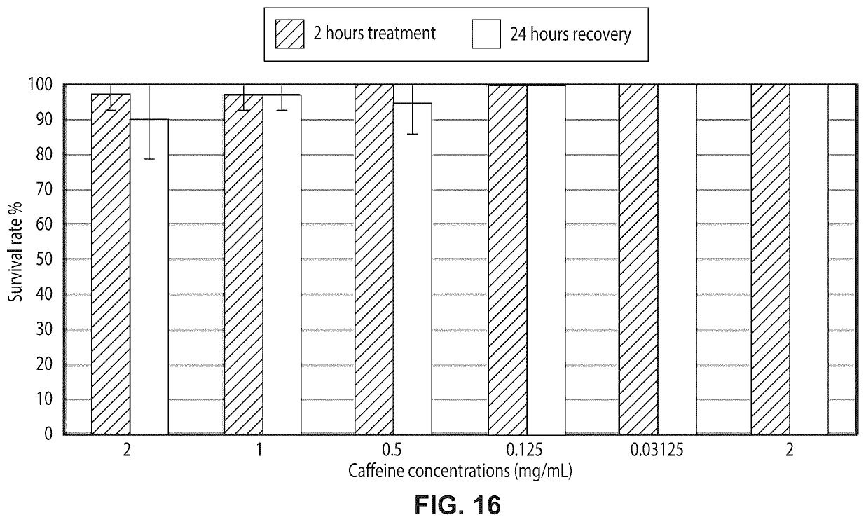

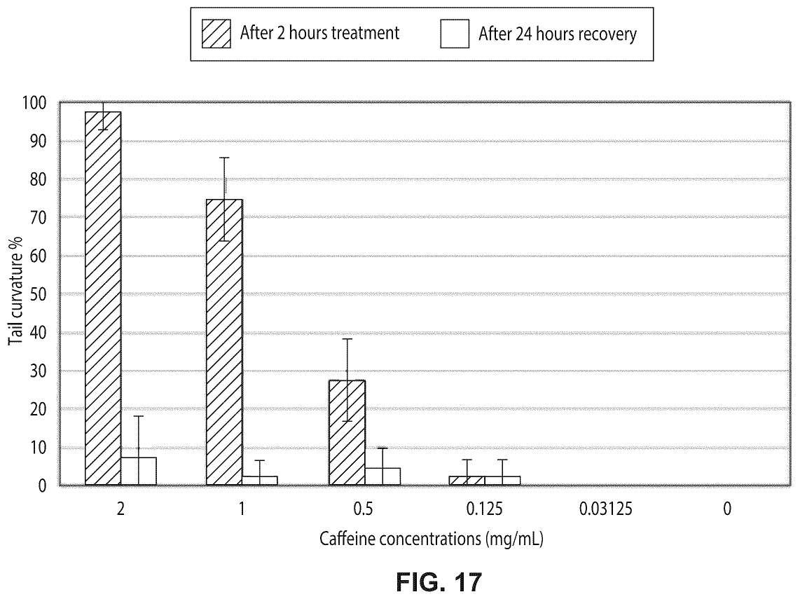

For the experiment, the zebrafish embryos were treated with caffeine for 2 hours following 24 hours recovery in E3 buffer using a schedule shown in FIG. 15 and the survival rate or tail curvature was found as a function of concentration as shown in FIG. 16 and FIG. 17.

Adult wild type zebrafish (EKW line) were randomly paired a night before mating and spawning. The eggs were collected using a sieve and then rinsed with the E3 buffer to filter out the debris and waste. The collected eggs were cultured in a Petri dish filled with E3 buffer at 28.5° C. The unfertilized eggs and dead embryos were sorted before/after the incubation. The chorions of 24 hpf zebrafish embryos were manually peeled off using micro tweezers (World Precision Instruments, Inc) under a stereomicroscope about 2 hours before the tests.

The dechorionated zebrafish embryos were then treated with different caffeine concentrations in 24 well plates (10 embryos per well) and different concentration gradients in CGG (26 embryos per CGG) for 2 hours. The caffeine was replaced by the E3 buffer after the 2-hour treatment in both well plate and CGG. Next, the zebrafish embryo was recovered in the well plate and CGG for 24 hours. The flowrate in CGG during the 24 hours of recovery process was set to be 2 ml/min. The temperature is maintained at 28.5° C. throughout the test. Images and endpoints were taken and recorded after 2-hour treatment and 24-hour recovery, respectively following the scheme shown in FIG. 15. Finally, the dose-response analysis was performed in Prism (GraphPad Software).

The zebrafish embryos images were taken using a stereomicroscope with a digital camera (AmScope Inc., USA). An LED plate (AmScope Inc. USA) was used as the light source for the imaging. A MATLAB script was used to estimate the caffeine concentration levels at individual traps via trypan blue gradient images. Specifically, an intensity profile along the traps was generated by inputting the steady state concentration gradient images and the coordinates of the traps to the MATLAB script. On the intensity profile, the trypan blue intensities inside the traps are displayed as upper peaks while the gaps between traps are flat signals. The peak signals on the intensity profiles then convert to the caffeine concentrations using the concentration versus intensity standard curve for trypan/caffeine buffer. The light intensity at each trap was measured in 8 bits and the intensity of trypan was calculated as 255 light intensities.

During the static experiments, the zebrafish embryos were treated with 6 caffeine concentrations, 2 mg/mL, 1 mg/mL, 0.5 mg/ml, 0.125 mg/mL, 0.0325 mg/mL, and 0 mg/mL (control) for the 2 hours in the 24-well plate (10 embryos per well, N≥4). The survival rate and sublethal endpoints were recorded after the 2-hour treatment and 24-hour recovery (FIG. 16). Note the caffeine was also mixed with 0.04% trypan blue for the later comparison with CGG-based caffeine overdose test.

The zebrafish embryos showed a strong vitality in the 2-day experiment in which over 90% of zebrafish embryos survived at the end of the experiment and no significant survival rate drop was observed in all caffeine concentrations as seen in FIG. 16. This indicated the selected caffeine concentrations were unable to trigger lethal endpoints in the zebrafish embryos within the experimental period.

After the 2-hour caffeine treatment, the very first and most prominent sublethal endpoint found in the zebrafish embryos was the tail curvature (i.e., scoliosis). The frequency of the zebrafish embryo tail curvature was found to be concentration dependent as the tail curvature event became more frequent as the caffeine concentration increased as shown in FIG. 17. The degree of embryo tail curvature was also seen to be concentration dependent as pig-tail-like tail curvatures were usually found at high caffeine concentrations. In addition, most of the zebrafish embryos with tail curvature were found to be recovered to normal after 24 hours culturing in E3 buffer. Because of the early timing of tail curvature development, it was marked as the primary sublethal endpoint for the zebrafish embryo caffeine overdose effect.

Another phenotype that was detected after the 24-hour recovery is edema. Both yolk sac edema (YE) and pericardial edema (PE) were observed after 24 hours of recovery. However, the edema prevalence was low in the well plate and could only be found at the highest caffeine concentration, 2 mg/ml. Because of that, edema is considered as a secondary endpoint for the instant caffeine overdose treatment. Note YE and PE are not distinguished and were both classified as edema in this study.

Collectively, within the current experiment settings, the chosen caffeine concentrations were able to simulate the caffeine overdose scenarios in the zebrafish embryo. The tail curvature is identified as the primary sublethal endpoint for the zebrafish embryos after the short-term caffeine treatment in the well plate and is expected to be detected in CGG-based caffeine overdose tests as well.

By comparison, the CGG-based acute FET test for caffeine overdose did not use the iFET (Sublethal Fish Embryo Toxicity Index) due to the temporal features of the sublethal endpoints. Instead, “the most instant sublethal endpoint” during or after the caffeine treatment was targeted for the dose-response analysis.

During the acute FET test in CGG, the zebrafish embryos were treated under 3 steady state caffeine concentration gradients for 2 hours. Specifically, the caffeine concentration gradients were generated by using 75 μl/min, 100 μl/min, and 150 μl/min as the inner wall inlet flowrates (Q_{in_inner wall}). A 2 mg/mL caffeine/0.04% trypan blue mixture was perfused through the inner wall inlet of the fluidic chip and mixed with E3 buffer loaded at the outer wall inlet for the dynamic concentration gradient generation (N≥6). The outer wall flowrate (Q_{in_outer wall}) was kept constant at 300 μl/min. For positive control, 150 μl/min was used as inner wall inlet flowrate (Q_{in_inner wall}) and both inner wall and outer wall inlets were perfused with 2 mg/mL caffeine/0.04% trypan blue mixture.

The steady state caffeine concentration gradients for the 3 selected flowrates can cover a caffeine concentration range (i.e., from 0.69 mg/mL to 2 mg/mL) that is expected to induce abnormality in the zebrafish embryos during/after the 2-hour treatment. For the 24-hour recovery, the caffeine was first flushed out of the CGG by the E3 buffer after the 2-hour treatment. The E3 buffer then circulates in the CGG system at 2 mL/min for the 24-hour recovery.

In terms of embryo vitality, the zebrafish embryos cultured in the CGG had a survival rate that is close to the zebrafish embryo tested in the well plate throughout the experiment without any significant drop between the first day and second day monitoring.

As expected, the tail curvature identified in the well plate results was also observed in the zebrafish embryos tested in the CGG. The overall tail curvature events were found elevated when the flowrate at the inner wall inlet was increased. No significant difference in overall tail curvature rate was found between the CGG positive control and the highest caffeine concentration (i.e., 2 mg/mL) treatment group in well plate.

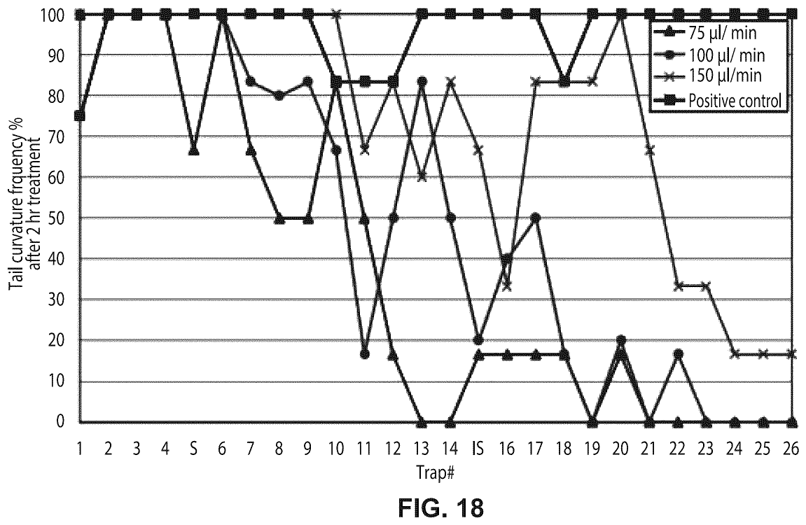

The zebrafish embryo tail curvature frequency distribution in the CGG was found following the caffeine concentration gradient as the tail curvature events were more frequent in the upstream traps as shown in FIG. 18. Moreover, when the overall caffeine concentration was elevated by increasing the flowrate at the inner wall inlet, the tail curvature occurrences shifted towards the downstream traps. Consistent with what was found in well plate-based tests (data not shown), the degree of zebrafish embryo tail curvature also seemed to correlate with the caffeine concentration as the “severe tail curvatures” such as pig-tail were often found at the upstream traps.

Overall, the sublethal endpoint, tail curvature, could be easily detected and parallelly compared in the CGG during/after the 2-hour caffeine treatment. However, the embryos preserved with the tail curvature after 24-hour recovery were found difficult to identify. The folded body postures from fluid flow were also observed in some of the zebrafish embryos cultured in the CGG on day 2. Crossflow may be the cause of the abnormal folded body postures because the shear stress was already reduced to a level that is close to what has been previously reported as a low shear stress level. In the current trap dimension, the zebrafish embryos would slightly outgrow the trap after 48 hpf and some of their body may be exposed to the flow at the main channel (i.e., continuously “hit” by the main channel flow) and trigger the abnormal postures. Due to the ambiguity of zebrafish embryo's tail postures, the attempts to quantify the embryos preserved with the tail curvature may cause false positive results after 24-hour recovery. Therefore, the measurements for zebrafish embryos with tail curvature were excluded in the CGG on day 2.

In addition to the tail curvature, edema was found surprisingly prevalent in the zebrafish embryos cultured in the CGG after 24 hour-recovery. In contrast to the tail curvature, the development of edema in the zebrafish embryos seemed more sensitive to the flowthrough environment as the overall frequency of edema occurrences was significantly higher in the CGG than in the well plate. The increased caffeine concentration level was also found to affect the edema development as the occurrence of edema increased when using higher inner wall inlet flowrates (i.e., more caffeine introduced).

As demonstrated above, during the 2-hour treatment, the zebrafish embryos developed tail curvature as a response to the different caffeine concentrations. To analyze how the tail curvature is affected by the different caffeine concentrations during the 2-hour treatment window, the dose-response curves for the 3 steady state caffeine concentration gradients as well as the well plate-based caffeine toxicity test were generated for comparison. Due to the limitations in the experimental concentration measurements in the CGG, the concentrations from both simulation estimations and experimental measurements were used to generate the dose-response curve for the 3 steady state caffeine concentration gradients. For comparison, the top and bottom for all the dose-response curves were constrained as 100% and 0%, respectively.

The absolute EC50 (50% effective concentration) for the caffeine during the 2-hour well plate based acute toxicity test was found to be 0.6904 mg/ml. For the CGG-based test, when using the simulated concentrations, the absolute EC50 for the 3 caffeine concentration gradients were found to be 1.175 mg/mL, 1.220 mg/mL, and 1.208 mg/mL, from lowest to highest inner wall inlet flowrates. Similarly, the absolute EC50 values were found to be 0.9932 mg/mL, 1.080 mg/mL, and 0.9869 mg/mL, from lowest to highest inner wall inlet flowrates, for the 3 tested gradients when inputting the measured concentrations. All the absolute EC50 values found in the CGG are higher than the absolute EC50 value for the well plate-based method which indicates the caffeine is less effective to trigger the tail curvature endpoint in the CGG than in the well plate during the 2-hour treatment. In addition, the hillslope values for the dose-response curves generated by the CGG are greater than the hillslope of well plate-based dose-response curves. The steeper dose-curves in the CGG suggested the zebrafish embryo is more concentration sensitive to the caffeine in the CGG than in the well plate during the 2-hour treatment. Furthermore, the absolute EC50 values and hillslope values found in CGG are close to each other among the 3 tested caffeine concentration gradients which indicates an excellent consistency for CGG-based acute caffeine FET tests.

CGG has proven to be feasible in performing short-term zebrafish embryo caffeine overdose studies. The tail curvature, which was observed in the well plate-based test, was also observed in the CGG and was identified as the primary sublethal endpoint for the acute caffeine overdose effect.

In addition, the zebrafish embryos were found to have a changing response to the different caffeine concentrations in the CGG which is consistent with the observations in the well plate. The CGG was found to be an effective tool in real-time embryo monitoring, parallel phenotype comparison, as well as high resolution dose-response analysis. Moreover, the caffeine dose-response curves for the embryo tail curvature were able to be obtained by replicating the high-resolution acute FET tests in the CGG (i.e., 6 replicates per gradient).

Comparisons between the caffeine dose-response curves generated by the CGG-based and well plate-based methods have shown that the zebrafish embryos have stronger caffeine tolerance (i.e., larger EC50), yet are more caffeine concentration sensitive (i.e., larger hillslope) in the CGG than in the well plate. This has not been reported by similar studies conducted in ZOC or well plate processes which can be useful in understanding the mechanism of caffeine overdose effects.

### Example 6

“One embryo per trap” is the most significant advantage for the CGG-based phenotype screening, however it is a disadvantage for concentration dependent tests as one embryo only corresponds to one concentration. This lowers the throughput and leads to the binary results at each concentration level.

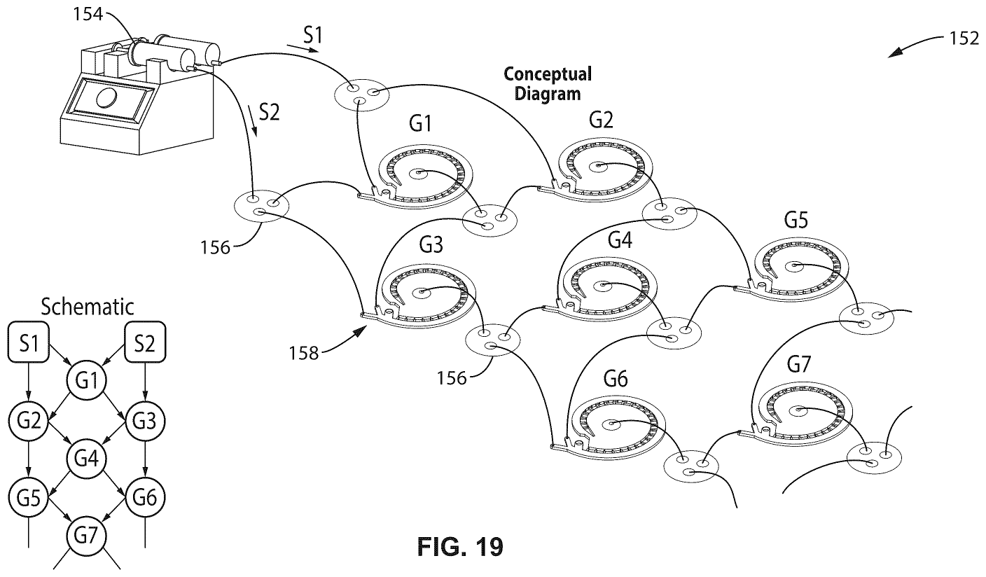

To address sample size limitation, in one embodiment, multiple CGGs may be connected in parallel using flow splitters such as a 3-way valve (i.e., 2-way flow splitter) as shown conceptually in FIG. 19. In this case, the sample size for each concentration is dependent on the capacity of the flow splitter and the number of flow splitters used. In another embodiment, the ZOC may be integrated with other concentration gradient generators (e.g., the Christmas tree-like CGG) and only the ZOC may be used for downstream zebrafish embryos immobilization purpose (i.e., give up the concentration gradient generation feature).

In the embodiment 152 shown in FIG. 19, the syringe pumps 154, flow splitters 156 and the CGGs 158 may be connected in a certain order from lines S1 and S2, so that more concentration gradients can be generated at the downstream CGGs 158 to have a wider range of concentration gradients.

In another embodiment, applying discontinuous dynamic concentration gradient generation as well as utilizing the feature of Laplace pressure-based droplet formation could be used to increase size impacts of the embryos, and the potential large reagent consumption for the dynamic concentration generation.

Accordingly, CGG can be used in high resolution dose-response screening (i.e., sublethal endpoints detection and parallel comparison) and concentration control. However, the throughput of the CGG-based acute FET test may be low for individual concentration and can be further improved via parallel connection of CGGs.

From the description herein, it will be appreciated that the present disclosure encompasses multiple implementations of the technology which include, but are not limited to, the following:

A fluidic device apparatus, comprising: (a) a fluidic chip body with a central fluid collection reservoir defined by an inner wall of an arcuate circumferential channel, the channel open at one end to the central fluid collection reservoir with an opening, the inner wall of the arcuate channel having a plurality of particle traps; (b) at least one fluid inlet fluidly coupled to the arcuate channel; and (c) a fluid outlet fluidly coupled to the central fluid collection reservoir; (d) wherein individual particles in a flow of fluid through the arcuate annular channel are trapped in the particle traps.

The apparatus of any preceding or following implementation, wherein the arcuate channel forms a single loop spiral, the opening of the channel to the central fluid collection reservoir at a distal end of the channel.

The apparatus of any preceding or following implementation, wherein the opening of the arcuate channel has a diameter that is smaller than the diameter of the arcuate channel.

The apparatus of any preceding or following implementation, further comprising a flow controller disposed within the arcuate channel configured to reduce fluid flow from the arcuate channel into the central fluid collection reservoir.

The apparatus of any preceding or following implementation, wherein the particle traps of the inner wall of the arcuate channel have a trap body open to the channel and a narrower trap nozzle that is open to the central fluid collection reservoir, the trap configured to allow fluid to flow from the arcuate channel through the trap body and trap nozzle to the central fluid collection reservoir.

The apparatus of any preceding or following implementation, wherein the trap body open to the arcuate channel has walls that are perpendicular to a surface of the inner wall of the arcuate channel.

The apparatus of any preceding or following implementation, wherein the trap body open to the arcuate channel of has an opening with angular edges.

The apparatus of any preceding or following implementation, wherein the trap body open to the arcuate channel of has an opening with an angular edge and a rounded edge, the rounded edge facing a direction of fluid flow towards a distal end of the arcuate channel.

The apparatus of any preceding or following implementation, wherein the at least one fluid inlet comprises a first fluidic input fluidly coupled to a proximal end of the arcuate channel; a second fluidic input fluidly coupled to a proximal end of the arcuate channel; and a carrier fluid input fluidly coupled to a proximal end of the arcuate channel.

A system for separating and retaining particles, the system comprising: (a) a fluidic chip with an arcuate channel with an outer wall and an inner wall, the arcuate channel forming a single loop spiral, the inner wall of the channel defining a central fluid collection reservoir, the arcuate channel open at one end to the central fluid collection reservoir, the inner wall of the arcuate channel having a plurality of particle traps; at least one fluid inlet fluidly coupled to the arcuate channel; and a fluid outlet fluidly coupled to the central fluid collection reservoir; (b) a pump; (c) a fluidic circuit between the pump and the fluid outlet of the central fluid collection reservoir of the fluidic chip; and (d) a controller configured to control actuation of the pump.

The system of any preceding or following implementation, wherein the pump comprises a peristaltic pump and a pulse dampener.

The system of any preceding or following implementation, further comprising: a closed fluidic circuit between the fluid outlet and the fluid inlet of the fluidic chip; and wherein fluid flows through the fluid inlet, the arcuate channel, the fluid collection reservoir and the fluid outlet can be recycled.

The system of any preceding or following implementation wherein the controller is configured to control a pressure of fluid entering the fluid inlet and a pressure of the fluid exiting the fluid outlet within the collection reservoir; and wherein the controller can create a pressure differential between the arcuate channel and the central fluid collection reservoir.

The system of any preceding or following implementation, the pump further comprises a fluid heating element; a temperature sensor; and a fluid temperature control circuit.

The system of any preceding or following implementation, further comprising one or more syringe pumps fluidly coupled to the at least one fluid inlets of the fluidic chip; a fluidic circuit with valves fluidly coupled to and input and an output side of the pump that can switch between a close-loop pumping configuration and an open-loop pumping configuration, the input side valves of the fluidic circuit also coupled to a first waste tank and the output side valves coupled to a second waste tank; wherein open-loop pumping fluid flow through the fluid inlet, the arcuate channel, the fluid collection reservoir and the fluid outlet is directed to a waste tank; and wherein close-loop pumping fluid flow through the fluid inlet, the arcuate channel, the fluid collection reservoir and the fluid outlet can be recycled through the fluid inlet.

A method for particle separations in a fluid flow, the method comprising: (a) providing a fluidic chip with an arcuate annular channel defined by an outer wall and an inner wall, the channel open at one end to a central fluid collection reservoir, the inner wall of the channel having a plurality of particle traps, at least one fluid inlet fluidly coupled to the arcuate channel; and a fluid outlet inlet fluidly coupled to the central fluid collection reservoir; (b) flowing a fluid with particles for separation through the fluid inlet and arcuate channel; and (c) trapping individual particles within the particle traps from the fluid flow by controlling the flow of fluid into the channel and out of the collection reservoir.

The method of any preceding or following implementation, further comprising: dispensing a first fluid through a first fluid inlet to the arcuate channel; dispensing a second fluid through a second fluid inlet to the arcuate channel; mixing the first and second fluids with a carrier fluid in the arcuate channel to produce mixed fluids; and flowing the mixed fluids through the arcuate channel, traps and central fluid collection reservoir; wherein a gradient of mixed fluids is generated in the arcuate channel.

The method of any preceding or following implementation, further comprising flushing the arcuate channel, traps and central fluid collection reservoir with carrier fluid after flowing the mixed fluids through the chip.

The method of any preceding or following implementation, further comprising controlling fluid temperature of the fluid entering the fluid inlet of the chip.

The method of any preceding or following implementation, further comprising recycling particle containing fluid exiting the fluid outlet back to the fluid inlet of the fluidic chip.

As used herein, the term “implementation” is intended to include, without limitation, embodiments, examples, or other forms of practicing the technology described herein.

As used herein, the singular terms “a,” “an,” and “the” may include plural referents unless the context clearly dictates otherwise. Reference to an object in the singular is not intended to mean “one and only one” unless explicitly so stated, but rather “one or more.”

Phrasing constructs, such as “A, B and/or C”, within the present disclosure describe where either A, B, or C can be present, or any combination of items A, B and C. Phrasing constructs indicating, such as “at least one of” followed by listing a group of elements, indicates that at least one of these groups of elements is present, which includes any possible combination of the listed elements as applicable.

References in this disclosure referring to “an embodiment”, “at least one embodiment” or similar embodiment wording indicates that a particular feature, structure, or characteristic described in connection with a described embodiment is included in at least one embodiment of the present disclosure. Thus, these various embodiment phrases are not necessarily all referring to the same embodiment, or to a specific embodiment which differs from all the other embodiments being described. The embodiment phrasing should be construed to mean that the particular features, structures, or characteristics of a given embodiment may be combined in any suitable manner in one or more embodiments of the disclosed apparatus, system, or method.

As used herein, the term “set” refers to a collection of one or more objects. Thus, for example, a set of objects can include a single object or multiple objects.

Relational terms such as first and second, top and bottom, upper and lower, left and right, and the like, may be used solely to distinguish one entity or action from another entity or action without necessarily requiring or implying any actual such relationship or order between such entities or actions.

The terms “comprises,” “comprising,” “has”, “having,” “includes”, “including,” “contains”, “containing” or any other variation thereof, are intended to cover a non-exclusive inclusion, such that a process, method, article, apparatus, or system, that comprises, has, includes, or contains a list of elements does not include only those elements but may include other elements not expressly listed or inherent to such process, method, article, apparatus, or system. An element proceeded by “comprises . . . a”, “has a”, “includes . . . a”, “contains . . . a” does not, without more constraints, preclude the existence of additional identical elements in the process, method, article, apparatus, or system, that comprises, has, includes, contains the element.

As used herein, the terms “approximately”, “approximate”, “substantially”, “substantial”, “essentially”, and “about”, or any other version thereof, are used to describe and account for small variations. When used in conjunction with an event or circumstance, the terms can refer to instances in which the event or circumstance occurs precisely as well as instances in which the event or circumstance occurs to a close approximation. When used in conjunction with a numerical value, the terms can refer to a range of variation of less than or equal to ±10% of that numerical value, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%. For example, “substantially” aligned can refer to a range of angular variation of less than or equal to ±10°, such as less than or equal to ±5°, less than or equal to ±4°, less than or equal to ±3°, less than or equal to ±2°, less than or equal to ±1°, less than or equal to ±0.5°, less than or equal to ±0.1°, or less than or equal to ±0.05°.

Additionally, amounts, ratios, and other numerical values may sometimes be presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a ratio in the range of about 1 to about 200 should be understood to include the explicitly recited limits of about 1 and about 200, but also to include individual ratios such as about 2, about 3, and about 4, and sub-ranges such as about 10 to about 50, about 20 to about 100, and so forth.

The term “coupled” as used herein is defined as connected, although not necessarily directly and not necessarily mechanically. A device or structure that is “configured” in a certain way is configured in at least that way, but may also be configured in ways that are not listed.

Benefits, advantages, solutions to problems, and any element(s) that may cause any benefit, advantage, or solution to occur or become more pronounced are not to be construed as a critical, required, or essential feature or element of the technology described herein or any or all the claims.

In addition, in the foregoing disclosure various features may be grouped together in various embodiments for the purpose of streamlining the disclosure. This method of disclosure is not to be interpreted as reflecting an intention that the claimed embodiments require more features than are expressly recited in each claim. Inventive subject matter can lie in less than all features of a single disclosed embodiment.

The abstract of the disclosure is provided to allow the reader to quickly ascertain the nature of the technical disclosure. It is submitted with the understanding that it will not be used to interpret or limit the scope or meaning of the claims.

It will be appreciated that the practice of some jurisdictions may require deletion of one or more portions of the disclosure after the application is filed. Accordingly, the reader should consult the application as filed for the original content of the disclosure. Any deletion of content of the disclosure should not be construed as a disclaimer, forfeiture, or dedication to the public of any subject matter of the application as originally filed.

All text in a drawing figure is hereby incorporated into the disclosure and is to be treated as part of the written description of the drawing figure.

The following claims are hereby incorporated into the disclosure, with each claim standing on its own as a separately claimed subject matter.

Although the description herein contains many details, these should not be construed as limiting the scope of the disclosure, but as merely providing illustrations of some of the presently preferred embodiments. Therefore, it will be appreciated that the scope of the disclosure fully encompasses other embodiments which may become obvious to those skilled in the art.

All structural and functional equivalents to the elements of the disclosed embodiments that are known to those of ordinary skill in the art are expressly incorporated herein by reference and are intended to be encompassed by the present claims. Furthermore, no element, component, or method step in the present disclosure is intended to be dedicated to the public regardless of whether the element, component, or method step is explicitly recited in the claims. No claim element herein is to be construed as a “means plus function” element unless the element is expressly recited using the phrase “means for”. No claim element herein is to be construed as a “step plus function” element unless the element is expressly recited using the phrase “step for”.

What is claimed is:

1. A fluidic device apparatus, comprising:
(a) a fluidic chip body with a central fluid collection reservoir defined by an inner wall of an arcuate circumferential channel, said channel open at one end to the central fluid collection reservoir with an opening, said inner wall of said arcuate channel having a plurality of particle traps;
(b) at least one fluid inlet fluidly coupled to said arcuate channel; and
(c) a fluid outlet fluidly coupled to said central fluid collection reservoir;
(d) wherein individual particles in a flow of fluid through the arcuate annular channel are trapped in the particle traps.

2. The apparatus of claim 1, wherein said arcuate channel forms a single loop spiral, said opening of the channel to the central fluid collection reservoir at a distal end of the arcuate channel.

3. The apparatus of claim 1, wherein said opening of said arcuate channel has a diameter that is smaller than a diameter of the arcuate channel.

4. The apparatus of claim 3, further comprising a flow controller disposed within said arcuate channel configured to reduce fluid flow from the arcuate channel into the central fluid collection reservoir.

5. The apparatus of claim 1, wherein said particle traps of said inner wall of said arcuate channel have a trap body open to the channel and a narrower trap nozzle that is open to the central fluid collection reservoir, said trap configured to allow fluid to flow from the arcuate channel through the trap body and trap nozzle to the central fluid collection reservoir.

6. The apparatus of claim 5, wherein said trap body open to the arcuate channel has walls that are perpendicular to a surface of the inner wall of the arcuate channel.

7. The apparatus of claim 5, wherein said trap body open to the arcuate channel of has an opening with angular edges.

8. The apparatus of claim 5, wherein said trap body open to the arcuate channel of has an opening with an angular edge and a rounded edge, said rounded edge facing a direction of fluid flow towards a distal end of the arcuate channel.

9. The apparatus of claim 1, wherein said at least one fluid inlet comprises:
a first fluidic input fluidly coupled to a proximal end of the arcuate channel;
a second fluidic input fluidly coupled to a proximal end of the arcuate channel; and
a carrier fluid input fluidly coupled to a proximal end of the arcuate channel.

10. A system for separating and retaining particles, the system comprising:
(a) a fluidic chip with an arcuate channel with an outer wall and an inner wall, said arcuate channel forming a single loop spiral, said inner wall of said channel defining a central fluid collection reservoir, said arcuate channel open at one end to the central fluid collection reservoir, said inner wall of said arcuate channel having a plurality of particle traps; at least one fluid inlet fluidly coupled to said arcuate channel; and a fluid outlet fluidly coupled to said central fluid collection reservoir;
(b) a pump;
(c) a fluidic circuit between the pump and the fluid outlet of the central fluid collection reservoir of the fluidic chip; and
(d) a controller configured to control actuation of the pump.

11. The system of claim 10, wherein said pump comprises a peristaltic pump and a pulse dampener.

12. The system of claim 10, further comprising:
a closed fluidic circuit between the fluid outlet and the fluid inlet of the fluidic chip; and
wherein fluid flows through the fluid inlet, the arcuate channel, the fluid collection reservoir and the fluid outlet can be recycled.

13. The system of claim 10:
wherein said controller is configured to control a pressure of fluid entering the fluid inlet and a pressure of the fluid exiting the fluid outlet within the collection reservoir; and
wherein said controller can create a pressure differential between the arcuate channel and the central fluid collection reservoir.

14. The system of claim 10, said pump further comprising:
a fluid heating element;
a temperature sensor; and
a fluid temperature control circuit.

15. The system of claim 10, further comprising:
one or more syringe pumps fluidly coupled to said at least one fluid inlet of said fluidic chip;
a fluidic circuit with valves fluidly coupled to and input and an output side of the pump that can switch between a close-loop pumping configuration and an open-loop pumping configuration, said input side valves of said fluidic circuit also coupled to a first waste tank and said output side valves coupled to a second waste tank;
wherein open-loop pumping fluid flow through the fluid inlet, the arcuate channel, the fluid collection reservoir and the fluid outlet is directed to a waste tank; and
wherein close-loop pumping fluid flow through the fluid inlet, the arcuate channel, the fluid collection reservoir and the fluid outlet can be recycled through the fluid inlet.

16. A method for particle separations in a fluid flow, the method comprising:
(a) providing a fluidic chip with an arcuate annular channel defined by an outer wall and an inner wall, said channel open at one end to a central fluid collection reservoir, said inner wall of said channel having a plurality of particle traps, at least one fluid inlet fluidly coupled to said arcuate channel; and a fluid outlet inlet fluidly coupled to said central fluid collection reservoir;
(b) flowing a fluid with particles for separation through said fluid inlet and arcuate channel; and
(c) trapping individual particles within said particle traps from the fluid flow by controlling the flow of fluid into the channel and out of the collection reservoir.

17. The method of claim 16, further comprising:
dispensing a first fluid through a first fluid inlet to said arcuate channel;
dispensing a second fluid through a second fluid inlet to said arcuate channel;
mixing said first and second fluids with a carrier fluid in the arcuate channel to produce mixed fluids; and
flowing the mixed fluids through the arcuate channel, traps and central fluid collection reservoir;
wherein a gradient of mixed fluids is generated in said arcuate channel.

18. The method of claim 17, further comprising:
flushing said arcuate channel, traps and central fluid collection reservoir with carrier fluid after flowing said mixed fluids through the chip.

19. The method of claim 16, further comprising controlling fluid temperature of the fluid entering the fluid inlet of the chip.

20. The method of claim 16, further comprising recycling particle containing fluid exiting the fluid outlet back to the fluid inlet of the fluidic chip.
